(19) United States
(12) Patent Application Publication
(10) Pub. No.: US 2009/0269403 A1
(43) Pub. Date: Oct. 29, 2009

(54) ORAL CONTRACEPTIVE DOSAGE FORMS AND METHODS OF MAKING SUCH DOSAGE FORMS

(76) Inventors: Ze'ev Shaked, San Antonio, TX (US); Justin R. Hughey, Round Rock, TX (US)

Correspondence Address: MEYERTONS, HOOD, KIVLIN, KOWERT & GOETZEL, P.C., P.O. BOX 398, AUSTIN, TX, 78767-0398, US

(21) Appl. No.: 12/429,009

(22) Filed: Apr. 23, 2009

Related U.S. Application Data
(60) Provisional application No. 61/047,505, filed on Apr. 24, 2008.
(60) Provisional application No. 61/061,041, filed on Jun. 12, 2008.
(60) Provisional application No. 61/116,560, filed on Nov. 20, 2008.

(51) Int. Cl. A61K 9/30; A61K 31/566

(52) U.S. Cl. 424/480; 514/170; 424/475

(57) ABSTRACT

Disclosed herein are oral dosage forms and methods of their use, in particular oral dosage systems for the delivery of drugs for use as a female oral contraceptive. In an embodiment, an oral dosage form includes a progestogen dispersed in an enteric polymer and an estrogen.

### PRIORITY CLAIM

This application claims priority to U.S. Provisional Patent Application No. 61/047,505, filed on Apr. 24, 2008, entitled “Oral Female Contraceptive Composition”; and U.S. Provisional Patent Application No. 61/061,041, filed on Jun. 12, 2008, entitled “Oral Female Contraceptive Composition”; and U.S. Provisional Patent Application No. 61/116,560, filed on Nov. 20, 2008, entitled “Oral Contraceptive Dosage Forms and Methods of Making Such Dosage Forms,” all of which are incorporated herein by reference.

### BACKGROUND OF THE INVENTION

1. Field of the Invention

The invention generally relates to oral dosage forms and methods of their use, in particular oral dosage systems for the delivery of drugs for use as a female oral contraceptive.

2. Description of the Relevant Art

Combined oral contraceptive pills, (e.g., oral contraceptives that include a combination of a gestagen and an estrogen component) were developed to inhibit normal fertility in women. Such pills inhibit follicular development and prevent ovulation as their primary mechanism of action. Combined oral contraceptive pills are favored over oral contraceptives that include a single dosage (e.g., a gestagen), due to a reduced incidence of breakthrough bleeding and various side effects.

Many of the side effects associated with oral contraceptive pills are due to the use of hormones to regulate the reproductive functions of women. Some of the potential side effects include: depression, vaginal discharge, changes in menstrual flow, breakthrough bleeding, nausea, vomiting, headaches, changes in the breasts, changes in blood pressure, loss of scalp hair, skin problems and skin improvements, increased risk of deep venous thrombosis (DVT) and pulmonary embolism, stroke and myocardial infarction (heart attack). The incidence of various side effects appears to be related, to some extent, on the dosage of both the gestagen and estrogen components. By minimizing the amount of one or both of these compounds in an oral contraceptive pill many of the known side effects may be reduced or eliminated. One of the most commonly used combined oral contraceptives includes a combination of 6β,7β,15β, 16β-dimethylene-3-oxo-17a-pregn-4-ene-21,17-carbolactone (drospirenone) and 17α-ethinylestradiol(ethinylestradiol). Oral dosage forms including drospirenone and ethinylestradiol are disclosed in U.S. Pat. No. 6,787,531 to Hilman et al., which is incorporated herein by reference. Drospirenone is only sparingly soluble in water at various pH values. The low aqueous solubility of drospirenone reduces its effectiveness due to poor bioavailability. Additionally, under acidic conditions (such as encountered in the gastric environment) drospirenone undergoes isomerization to a form that is inactive. The combination of poor aqueous solubility and potential for isomerization makes the use of low dosage forms of drospirenone difficult. This leads to the use of higher than necessary dosages of drospirenone to counteract the inactivation and slow absorption of the active form. It is therefore desirable to develop low dosage drospirenone formulations to reduce the incidence of side effects and cost associated with making higher dosage oral contraceptives.

One solution to the problem of drospirenone isomerization that has been attempted by researchers is to use an enteric coating for drospirenone to inhibit isomerization of drospirenone in the gastric environment. Studies of the bioavailability of enteric coated oral contraceptive formulations that include drospirenone showed significant intra- and inter-individual differences regarding bioavailability after administration of enteric coated formulations. While enteric coatings provide a solution to the isomerization problem of drospirenone, it is desirable that formulations are developed that are resistive to the gastric environment while providing consistent and appropriate bioavailability of the oral contraceptive agents.

### SUMMARY OF THE INVENTION

In certain embodiments, oral dosage forms for the delivery of drugs for use as a female oral contraceptive include a progestogen dispersed in an enteric polymer and an estrogen. The oral dosage form may include between about 0.5 mg to about 50 mg of the progestogen. The oral dosage form may include between about 0.01 mg to about 0.1 mg of the estrogen. Suitable enteric polymers for the oral dosage form include, but are not limited to: phthalate based polymers (e.g., cellulose acetate phthalate, polyvinyl acetate phthalate, methylcellulose phthalate, hydroxypropyl methylcellulose phthalate, or ethylhydroxycellulose phthalate), hydroxypropyl methylcellulose based polymers (e.g., hydroxypropyl methylcellulose acetate succinate), and acrylic acid-based polymer, a methacrylic acid based polymer, or an acrylic acid-methacrylic acid based copolymer. The oral dosage form may include suitable plasticizers (e.g., polycarboxylic acids) and other known excipients (e.g., pore formers). In one embodiment, the progestogen is drospirenone and the estrogen is ethinylestradiol. In another embodiment, the progestogen is drospirenone and the estrogen is a nitrated estrogen derivative.

In certain embodiments, oral dosage forms for the delivery of drugs for use as a female oral contraceptive may be in the form of a tablet that includes particles of a progestogen dispersed in an enteric polymer and an estrogen. The tablet may further include a coating layer that at least partially covers the tablet and that includes an enteric polymer. The oral dosage form may include between about 0.5 mg to about 50 mg of the progestogen. The oral dosage form may include between about 0.01 mg to about 0.1 mg of the estrogen. In one embodiment, the progestogen is drospirenone and the estrogen is ethinylestradiol. In another embodiment, the progestogen is drospirenone and the estrogen is a nitrated estrogen derivative.

In certain embodiments, oral dosage forms for the delivery of drugs for use as a female oral contraceptive may be in the form of a tablet that includes particles of a progestogen dispersed in an enteric polymer. The tablet further includes a coating layer, that at least partially covers the tablet and that includes an estrogen. The oral dosage form may include between about 0.5 mg to about 50 mg of the progestogen. The oral dosage form may include between about 0.01 mg to about 0.1 mg of the estrogen. The coating layer may include the estrogen dispersed in a polymer (e.g., an enteric polymer). In one embodiment, the progestogen is drospirenone and the estrogen is ethinylestradiol. In another embodiment, the progestogen is drospirenone and the estrogen is a nitrated estrogen derivative.

In certain embodiments, oral dosage forms for the delivery of drugs for use as a female oral contraceptive include drospirenone dispersed in an enteric polymer as a monolithic, solidified. The oral dosage form further includes a coating layer, that at least partially covers the tablet and that includes an estrogen. The oral dosage form may include between about 0.5 mg to about 50 mg of the progestogen. The oral dosage form may include between about 0.01 mg to about 0.1 mg of the estrogen. The coating layer may include the estrogen dispersed in a polymer (e.g., an enteric polymer). In one embodiment, the progestogen is drospirenone and the estrogen is ethinylestradiol. In another embodiment, the progestogen is drospirenone and the estrogen is a nitrated estrogen derivative.

In certain embodiments, a method of producing a contraceptive state in a subject includes administering to a subject an oral dosage form that includes an effective amount of a progestogen dispersed in an enteric polymer and an effective amount of an estrogen. In one embodiment, the oral dosage form includes an effective amount of a progestogen and an effective amount of an estrogen in a tablet form, wherein the tablet includes progestogen particles and the progestogen particles include the progestogen dispersed in an enteric polymer. In another embodiment, the oral dosage form includes an effective amount of a progestogen and an effective amount of an estrogen in a tablet form. The tablet includes progestogen particles, the progestogen particles including a progestogen dispersed in an enteric polymer. The tablet further includes a coating covering at least a portion of the tablet, the coating comprising an estrogen. In another embodiment, the oral dosage form includes the progestogen dispersed in an enteric polymer as a monolithic, solidified form and a coating covering at least a portion of the monolithic, solidified form, the coating including the estrogen.

In certain embodiments, an oral dosage form as described above, is formed by forming a mixture of an enteric polymer and a progestogen; heating at least a portion of the enteric polymer to form a heated mixture of enteric polymer and the progestogen; cooling the heated mixture; and combining the cooled mixture with an estrogen to form the oral dosage form.

In another embodiment, an oral dosage form as described above, is formed by forming a forming a mixture of an enteric polymer and a progestogen; heating at least a portion of the enteric polymer to a temperature at or above the glass transition temperature of the enteric polymer to form a heated mixture of enteric polymer and the progestogen; permitting the heated mixture to solidify as a solid mass; preparing progestogen particles from the solid mass; and forming the oral dosage tablet from the progestogen particles and an estrogen.

In another embodiment, an oral dosage form as described above, is formed by forming a forming a mixture of an enteric polymer and a progestogen; heating at least a portion of the enteric polymer to a temperature at or above the glass transition temperature of the enteric polymer to form a heated mixture of enteric polymer and the progestogen; permitting the heated mixture to solidify as a solid mass; preparing progestogen particles from the solid mass; and forming the oral dosage tablet from the progestogen particles and an estrogen.

In another embodiment, an oral dosage form as described above, is formed by forming a mixture of an enteric polymer and a progestogen; heating at least a portion of the enteric polymer to a temperature at or above the glass transition temperature of the enteric polymer to form a heated mixture of enteric polymer and the progestogen; permitting the heated mixture to solidify as a solid mass; preparing progestogen particles from the solid mass; forming a tablet from the progestogen particles; and forming a coating on at least a portion of the tablet, the coating comprising an estrogen.

In another embodiment, an oral dosage form as described above, is formed by forming a mixture of an enteric polymer and a progestogen; heating at least a portion of the enteric polymer to a temperature at or above the glass transition temperature of the enteric polymer to form a heated mixture of enteric polymer and progestogen; permitting the heated mixture to solidify as a solid mass to form a monolithic, solidified unit; and forming a coating on at least a portion of the monolithic, solidified unit, the coating comprising an estrogen.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts the dissolution profiles for drospirenone particles in various aqueous solutions;

FIG. 2 depicts the particle size distribution of the Drospirenone Process Intermediate and the EE Granulation Intermediate;

FIG. 3 depicts the drug release profiles for drospirenone/ethinylestradiol tablets; and

FIG. 4. depicts additional drug release profiles for drospirenone/ethinylestradiol tablets.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

Embodiments described herein relate to oral dosage forms that are designed to inhibit ovulation in a human female by the administration of a combination of a progestogen and an estrogen. Furthermore, the embodiments described herein are directed to methods of formulating such oral dosage forms. Additionally, embodiments described herein provide methods of administering such oral dosage forms.

Progestogens include, but are not limited to: 17α-17-hydroxy-11-methylene-19-norpregna-4,15-dien-20-yn-3-one, 17α-ethynyl-19-nortestosterone, 17α-ethynyltestosterone, 17-deacetylnorgestimate, 19-nor-17-hydroxyprogesterone, 19-norprogesterone, 3β-hydroxydesogestrel, 3-ketodesogestrel (etonogestrel), acetoxypregnenolone, algestone acetophenide, allylestrenol, amgestone, anagestone acetate, chlormadinone, chlormadinone acetate, cyproterone, cyproterone acetate, d-17β-acetoxy-13β-ethyl-17α-ethynylgon-4-en-3-one oxime, demegestone, desogestrel, dienogest, dihydrogesterone, dimethisterone, drospirenone, dydrogesterone, ethisterone (pregneninolone, 17α-ethynyltestosterone), ethynodiol diacetate, etonogestrel, fluorogestone acetate, gastrinone, gestadene, gestodene, gestonorone, gestrinone, hydroxymethylprogesterone, hydroxymethylprogesterone acetate, hydroxyprogesterone, hydroxyprogesterone acetate, hydroxyprogesterone caproate, levonorgestrel (1-norgestrol), lynestrenol (lynoestrenol), mecirogestone, medrogestone, medroxyprogesterone, medroxyprogesterone acetate, megestrol, megestrol acetate, melengestrol, melengestrol acetate, nestorone, nomegestrol, norelgestromin, norethindrone (norethisterone) (19-nor-17α-ethynyltestosterone), norethindrone acetate (norethisterone acetate), norethynodrel, norgestimate, norgestrel (d-norgestrel and dl-norgestrel), norgestrienone, normethisterone, progesterone, promegestone, quingestanol, tibolone, and trimegestone.

Estrogens include, but are not limited to, estradiol (17β-estradiol), estridiol acetate, estradiol benzoate, estridiol cypionate, estridiol decanoate, estradiol diacetate, estradiol heptanoate, estradiol valerate, 17α-estradiol, estriol, estriol succinate, estrone, estrone acetate, estrone sulfate, estropipate (piperazine estrone sulfate), ethynylestradiol (17α-ethynylestradiol, ethinylestradiol, ethinyl estradiol, ethynyl estradiol), ethynylestradiol 3-acetate, ethynylestradiol 3-benzoate, mestranol, quinestrol, and nitrated estrogen derivatives.

Nitrated estrogen derivatives are described in U.S. Pat. No. 5,554,603 to Kim et al. which is incorporated herein by reference. Nitrated estrogen derivatives that may be used in combination with a progestogen include compounds having the structure:

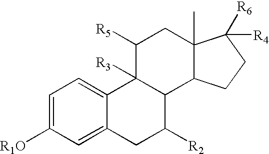

where R₁ is hydrogen, C₁-C₈ alkyl, cycloalkyl, or C₁-C₈ acyl;
R₂ is hydrogen or C₁-C₈ alkyl;
R₃ is hydrogen, hydroxy or C₁-C₈ alkyl;
R₄ is hydrogen or C₁-C₈ alkyl;
where each R₅ and R₆ is, independently, hydrogen or nitrate; and wherein at least one of R₅ and R₆ is a nitrate group.

In some embodiments, the nitrated estrogen derivative has the structure:

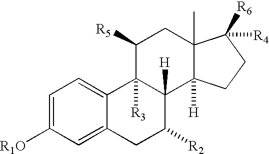

where R₁ is hydrogen, C₁-C₈ alkyl, cycloalkyl, or C₁-C₈ acyl;
R₂ is hydrogen or C₁-C₈ alkyl;
R₃ is hydrogen, hydroxy or C₁-C₈ alkyl;
R₄ is hydrogen or C₁-C₈ alkyl;
where each R₅ and R₆ is, independently, hydrogen or nitrate; and wherein at least one of R₅ and R₆ is a nitrate group.

A specific compound that may be used in combination with a progestogen in an oral contraceptive to inhibit ovulation in a female subject includes the compound (+)-3,11β,17β-trihydroxyestra-1,3,5(10)-triene 11,17-dinitrate ester, which has the structure:

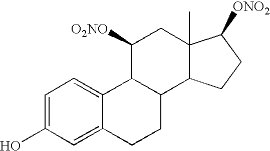

In some embodiments the oral dosage form is monolithic and substantially solid, that is it is formed as a unitary mass that is molded, cut, ground or otherwise formed in its final shape. In other embodiments, the oral dosage form may be an aggregate or composite of individual solid particulates, pellets, beads microspheres or the like formed into a tablet or disposed in a capsule.

The phrase “oral dosage form” as used herein refers to pharmaceutical compositions formed as tablets, caplets and the like that are swallowed substantially intact when used as intended. Films, wafers and the like which are not intended to be swallowed substantially intact are not contemplated embodiments of oral dosage forms.

Oral dosage forms described herein for use as an oral contraceptive include a progestogen and an estrogen in amounts effective to inhibit ovulation in a female subject. An oral dosage form may include, but is not limited to, between about 0.5 mg to about 50 mg, between about 1 mg to 30 mg, or between about 2 mg to 10 mg of a progestogen. An oral dosage form includes, but is not limited to, 1 mg, 1.5 mg, 2 mg, 2.5 mg, 3 mg, 3.5 mg, 4 mg, 4.5 mg, or 5 mg of a progestogen.

In addition to a progestogen, an oral dosage form may further include an estrogen. An oral dosage form may include, but is not limited to, between about 0.01 mg to about 0.1 mg, between about 0.015 mg to 0.075 mg, or between about 0.02 mg to 0.05 mg of an estrogen. An oral dosage form includes, but is not limited to, 0.01 mg, 0.015 mg, 0.02 mg, 0.025 mg, 0.03 mg, 0.035 mg, 0.04 mg, 0.045 mg, 0.05 mg, or 0.06 mg of an estrogen.

In certain embodiments, an oral dosage form includes an enteric polymer in which a progestogen, estrogen, or both therapeutic agents are dispersed. In an embodiment, the enteric polymer is a fusible, thermoplastic or thermosetting material, typically a resin or polymer. An enteric polymer may make up about 20% to about 99.9% of the oral dosage form by weight, or at least about 30%, at least about 40%, or at least about 50% of the oral dosage form by weight.

As used herein the term “enteric polymer” is a polymer that is substantially insoluble and/or impermeable to the acidic environment of the stomach (e.g., pH of about 1-3) and soluble or permeable in the environment of the intestine (pH of about 5-7). An enteric polymer, as defined herein, releases less than 10% of a therapeutic agent dispersed in the enteric polymer after 2 hours of stirring the dispersion in a 0.1 N HCl solution and releases more than about 10% of a therapeutic agent dispersed in the enteric polymer after about 2 hours stirring in a pH 6.8 phosphate buffer solution.

As used herein the term “dispersed”, with respect to a polymer matrix, means that a compound is substantially evenly distributed through the polymer, either as a solid suspension in the polymer or dissolved within the polymer matrix. The term “particle dispersion,” as used herein refers to a suspension of the compound particles homogenously distributed in the polymer. The term “molecular dispersion,” as used herein refers to the dissolution of the compound in the polymer. For purposes of this disclosure, a dispersion may be characterized as a particle dispersion if particles of the compound are visible in the polymer at a magnification of about 100× under regular and polarized light. A molecular dispersion is characterized as a dispersion in which substantially no particles of the compound are visible in the polymer at a magnification of 100× under regular and polarized light.

The release characteristics of the oral dosage form can be determined in vitro using simulated gastric or intestinal fluids, but is preferably determined in vivo by monitoring blood levels of the therapeutic agent in subjects that have ingested the oral dosage form. Methods of determining the in vivo and in vitro release of therapeutic agents from oral dosage forms are well-known to those skilled in the art.

Enteric polymers may include polymers with acidic functional groups. Enteric polymers may solubilize or become permeable in an aqueous solution at a pH of greater than 5. Examples of enteric polymers include cellulose based enteric polymers, vinyl based enteric polymers, and acrylic acid-methacrylic acid based enteric polymers. As used herein, the phrase “acrylic acid-based polymers” refers to any polymer that includes one or more repeating units that include and/or are derived from acrylic acid. As used herein, the phrase “methacrylic acid-based polymers” refers to any polymer that includes one or more repeating units that include and/or are derived from methacrylic acid.

Examples of cellulose based enteric polymers include, but are not limited to, cellulose acetate phthalate (CAP), cellulose acetate succinate, methylcellulose phthalate, ethylhydroxycellulose phthalate, hydroxypropyl methylcellulose phthalate (HPMCP), hydroxypropyl methylcellulose acetate succinate (HPMCAS), cellulose acetate butyrate, and cellulose acetate trimaleate.

Examples of vinyl based enteric polymers include, but are not limited to, polyvinyl acetate phthalate (PVAP), polyvinylbutyrate acetate, polyvinyl acetate, vinyl acetate-maleic anhydride copolymer, and polyvinyl acetal diethylaminoacetate.

Examples of acrylic acid-methacrylic acid based enteric polymers include, but are not limited to, Eudragit® L30-D55, Eudragit® FS 30D, Eudragit® L 12.5, Eudragit® L 12.5P, Eudragit® L100, Eudragit® L100-55, Eudragit® L-30, Eudragit® LD-55, Eudragit® S 12.5, Eudragit® S 12.5P, Eudragit®V S100, Eudragit® S100-55, Eudragit® NE 30D, Eudragit® RL 12.5, Eudragit® RL 100, Eudragit® RL PO, Eudragit® RL 30D, Eudragit® RS 12.5, Eudragit® RS 100, Eudragit® RS PO, and Eudragit® RS 30D.

One or more water-insoluble polymers may be combined with one or more enteric polymers to form an oral dosage form. Examples of pharmaceutically-acceptable, water-insoluble polymers include, but are not limited to acrylic acid-based polymers, methacrylic acid based polymers, and acrylic acid-methacrylic acid based copolymers. As used herein, the phrase “acrylic acid-based polymers” refers to any polymer that includes one or more repeating units that include and/or are derived from acrylic acid. As used herein, the phrase “methacrylic acid-based polymers” refers to any polymer that includes one or more repeating units that include and/or are derived from methacrylic acid. Derivatives of acrylic acid and methacrylic acid include, but are not limited to, alkyl ester derivatives, alkylether ester derivatives, amide derivatives, alkyl amine derivatives, anhydride derivatives, cyanoalkyl derivatives, and amino-acid derivatives. Examples of acrylic acid-based polymers, methacrylic acid based polymers, and acrylic acid-methacrylic acid based copolymers include, but are nor limited to Eudragit® L100, Eudragit® L100-55, Eudragit® L 30 D-55, Eudragit® S100, Eudragit® 4135F, Eudragit® RS, acrylic acid and methacrylic acid copolymers, methyl methacrylate polymers, methyl methacrylate copolymers, polyethoxyethyl methacrylate, polycyanoethyl methacrylate, aminoalkyl methacrylate copolymer, polyacrylic acid, polymethacrylic acid, methacrylic acid alkylamine copolymer, polymethyl methacrylate, polymethacrylic acid anhydride, polyalkylmethacrylate, polyacrylamide, and polymethacrylic acid anhydride and glycidyl methacrylate copolymers.

Further examples of pharmaceutically-acceptable, water-insoluble polymers include, but are not limited to, alkylcelluloses such as ethylcellulose, methylcellulose, and calcium carboxymethyl cellulose, and polyesters, waxes, shellac, zein, or the like.

In further embodiments, in addition to containing 20 to 99.9% by weight of one or more enteric polymers, the oral dosage forms may further include one or more pharmaceutically-acceptable hydrophilic matrix materials including water-soluble polymers such as polyethylene oxide (PEO), ethylene oxide-propylene oxide co-polymers, polyethylene-polypropylene glycol (e.g. poloxamer), carbomer, polycarbophil, chitosan, polyvinyl pyrrolidone (PVP), polyvinyl alcohol (PVA), hydroxyalkyl celluloses such as hydroxypropyl cellulose (HPC), hydroxyethyl cellulose, hydroxymethyl cellulose and hydroxypropyl methylcellulose, carboxymethyl cellulose, sodium carboxymethyl cellulose, methylcellulose, hydroxyethyl methylcellulose, hydroxypropyl methylcellulose, polyacrylates such as carbomer, polyacrylamides, polymethacrylamides, polyphosphazines, polyoxazolidines, polyhydroxyalkylcarboxylic acids, alginic acid and its derivatives such as carrageenate alginates, ammonium alginate and sodium alginate, starch and starch derivatives, polysaccharides, carboxypolymethylene, polyethylene glycol, natural gums such as gum guar, gum acacia, gum tragacanth, karaya gum and gum xanthan, povidone, gelatin or the like.

For purposes of the present disclosure, a matrix material is considered hydrophilic and a polymer is considered to be water-soluble if it is more than sparingly soluble as defined by USP 29/NF 24, that is if according to USP 29/NF 24 the matrix material or polymer is classified as “soluble” or “very soluble.”

Preferred materials used to produce an oral dosage form will be pharmaceutically acceptable materials, such as those indicated to be generally regarded as safe (“GRAS-certified”) or national formulary certified.

In preferred embodiments, a plasticizer is also combined with the enteric polymer to modify the properties of the polymer. Plasticizers interact with the enteric polymer resulting in a lower viscosity of the mixture during extrusion or molding. The result is that extrusion or injection molding of the oral dosage form can occur at lower temperatures, thereby reducing the possibility of thermally degrading the therapeutic agent (e.g., drospirenone or ethinylestradiol). The most suitable plasticizers are those that lower the glass transition temperature (Tg) of the enteric polymer. Plasticizers suitable for use with the compositions and methods disclosed herein include, but are not limited to, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, acetyl triethyl citrate, tributyl citrate and allyl glycolate. In addition to an enteric polymer and a therapeutic agent, compositions may also include one or more functional excipients such as lubricants, thermal lubricants, antioxidants, buffering agents, alkalinizing agents, disintegrants, binders, diluents, sweeteners, chelating agents, colorants, flavorants, surfactants, solubilizers, wetting agents, stabilizers, hydrophilic polymers, hydrophobic polymers, waxes, lipophilic materials, absorption enhancers, preservatives, absorbents, cross-linking agents, bioadhesive polymers, retardants, pore formers, osmotic agents and fragrance.

Lubricants or thermal lubricants useful as an excipient include, but are not limited to fatty esters, glyceryl monooleate, glyceryl monostearate, wax, carnauba wax, beeswax, vitamin E succinate, and a combination thereof.

As used herein, the term “antioxidant” is intended to mean an agent that inhibits oxidation and thus is used to prevent the deterioration of preparations by oxidation due to the presence of oxygen free radicals or free metals in the composition. Such compounds include, by way of example and without limitation, ascorbic acid (Vitamin C), ascorbyl palmitate, butylated hydroxyanisole (BHA), butylated hydroxytoluene (BHT), hypophosphorous acid, monothioglycerol, sodium ascorbate, sodium formaldehyde sulfoxylate, sodium metabisulfite, sodium bisulfite, vitamin E and its derivatives, propyl gallate and others known to those of ordinary skill in the art.

Binders are ingredients added to mixtures to provide adhesive qualities during and after formation of an oral dosage. Examples of binders include, but are not limited to: waxes such as beeswax; carnauba wax; microcrystalline wax and paraffin wax; cetyl palmitate; glycerol behenate; glyceryl palmitostearate; glyceryl stearate; hydrogenated castor oil; stearic acid; stearic alcohol; stearate 6000 WL1644; gelucire 50/13; polyethylene glycols (PEG) such as PEG 2000, PEG 3000, PEG 6000, PEG 8000, PEG 10000, PEG 20000; polyethylene oxide; polypropylene oxide; polyvinylpyrrolidone; polyvinylpyrrolidone-co-vinylacetate; acrylate-methacrylate copolymers; polyethylene; polycaprolactone; alkylcelluloses such as methylcellulose; hydroxyalkylcelluloses such as hydroxymethylcellulose, hydroxyethylcellulose, hydroxypropylcellulose, and hydroxybutylcellulose; hydroxyalkyl alkylcelluloses such as hydroxyethyl methylcellulose and hydroxypropyl methylcellulose; starches, pectins; polylactic acid (PLA); polyglycolic acid (PLGA), polyesters (e.g., shellac); and polysaccharides such as cellulose, tragacanth, gum arabic, guar gum, and xanthan gum.

A buffering agent is used to resist change in pH upon dilution or addition of acid or alkali. Such compounds include, by way of example and without limitation, potassium metaphosphate, potassium phosphate, monobasic sodium acetate and sodium citrate anhydrous and dihydrate, salts of inorganic or organic acids, salts of inorganic or organic bases, and others known to those of ordinary skill in the art.

As used herein, the term “alkalizing agent” is intended to mean a compound used to provide alkaline medium for product stability. Such compounds include, by way of example and without limitation, ammonium carbonate, diethanolamine, monoethanolamine, potassium hydroxide, sodium borate, sodium carbonate, sodium bicarbonate, sodium hydroxide, triethanolamine and others known to those of ordinary skill in the art.

As used herein, the term “disintegrant” is intended to mean a compound used in solid dosage forms to promote the disruption of a solid mass (layer) into smaller particles that are more readily dispersed or dissolved. Exemplary disintegrants include, by way of example and without limitation, starches such as corn starch, potato starch, pre-gelatinized and modified starches thereof, sweeteners, clays, bentonite, microcrystalline cellulose (e.g., Avicel™), carboxymethylcellulose calcium, croscarmellose sodium, alginic acid, sodium alginate, cellulose polyacrilin potassium (e.g., Amberlite™), alginates, sodium starch glycolate, gums, agar, guar, locust bean, karaya, pectin, tragacanth, crospovidone and other materials known to one of ordinary skill in the art. A superdisintegrant is a rapidly acting disintegrant. Exemplary superdisintegrants include crospovidone and low substituted HPC. Exemplary chelating agents include EDTA, polyamines, derivatives thereof, and others known to those of ordinary skill in the art.

As used herein, the term “colorant” is intended to mean a compound used to impart color to solid (e.g., tablets) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and ferric oxide, red, other FD&C dyes and natural coloring agents such as grape skin extract, beet red powder, beta carotene, annato, carmine, turmeric, paprika, and other materials known to one of ordinary skill in the art. The amount of coloring agent used will vary as desired.

As used herein, the term “flavorant” is intended to mean a compound used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors that have been found to be particularly useful include commercially available orange, grape, cherry and bubble gum flavors and mixtures thereof. The amount of flavoring may depend on a number of factors, including the organoleptic effect desired. Flavors will be present in any amount as desired by those of ordinary skill in the art. Particular flavors are the grape and cherry flavors and citrus flavors such as orange.

Surfactants include soaps, synthetic detergents, and wetting agents. Suitable surfactants include cationic surfactants, anionic surfactants, non-ionic surfactants, and amphoteric surfactants. Examples of surfactants include Polysorbate 80; sorbitan monooleate; sodium lauryl sulfate (sodium dodecylsulfate); soaps such as fatty acid alkali metal salts, ammonium salts, and triethanolamine salts; cationic detergents such as dimethyl dialkyl ammonium halides, alkyl pyridinium halides, and alkylamine acetates; anionic detergents such as alkyl, aryl and olefin sulfonates, alkyl, olefin, ether and monoglyceride sulfates, and sulfosuccinates; nonionic detergents such as fatty amine oxides, fatty acid alkanolamides, and poly(oxyethylene)-block-poly(oxypropylene) copolymers; and amphoteric detergents, for example, alkyl β-aminopropionates and 2-alkylimidazoline quaternary ammonium salts; wetting agents such as, glycerin, proteins, and peptides; water miscible solvents such as glycols; and mixtures thereof.

Solubilizers include cyclodextrins, povidone, combinations thereof, and others known to those of ordinary skill in the art.

Exemplary hydrophilic polymers which can be a primary or secondary polymeric carrier that can be included in the composition include poly(vinyl alcohol) (PVA), polyethylene-polypropylene glycol (e.g. poloxamer), carbomer, polycarbophil, or chitosan. Hydrophilic polymers include, but are not limited to, one or more of, carboxymethylcellulose, hydroxyalkyl celluloses such as hydroxypropyl cellulose, hydroxyethyl cellulose, hydroxyethyl methyl cellulose, and hydroxymethyl cellulose, methylcellulose, natural gums such as gum guar, gum acacia, gum tragacanth, or gum xanthan and povidone. “Hydrophilic polymers” also include polyethylene oxide, sodium carboxymethycellulose, carboxypolymethylene, polyethylene glycol, alginic acid, gelatin, polyvinyl alcohol, polyvinylpyrrolidones, polyacrylamides, polymethacrylamides, polyphosphazines, polyoxazolidines, poly(hydroxyalkylcarboxylic acids), carrageenate alginates, carbomer, ammonium alginate, sodium alginate, or mixtures thereof.

Exemplary hydrophobic polymers include alkylcelluloses, ethyl cellulose, Eudragit RS, waxes, polyesters, combinations thereof, and others known to those of ordinary skill in the art.

Exemplary waxes include carnauba wax, beeswax, microcrystalline wax and others known to one of ordinary skill in the art.

Exemplary absorption enhancers include dimethyl sulfoxide, Vitamin E PGS, sodium cholate and others known to one of ordinary skill in the art.

Preservatives include compounds used to prevent the growth of microorganisms. Suitable preservatives include, by way of example and without limitation, benzalkonium chloride, benzethonium chloride, benzyl alcohol, cetylpyridinium chloride, chlorobutanol, phenol, phenylethyl alcohol, phenylmercuric nitrate and thimerosal and others known to those of ordinary skill in the art.

Examples of absorbents include sodium starch glycolate (Explotab™, Primojel™); croscarmellose sodium (Ac-Di-Sol®); polyvinylpyrrolidone (PVP) (e.g., Polyplasdone™ XL 10); veegum; clays; alginates; alginic acid; carboxymethylcellulose calcium; microcrystalline cellulose (e.g., Avicel™); polacrillin potassium (e.g., Amberlite™); sodium alginate; corn starch; potato starch; pregelatinized starch; modified starch; cellulosic agents; montmorrilonite clays (e.g., bentonite); gums; agar: locust bean gum; gum karaya; pecitin; tragacanth; and other absorbents known in to those of ordinary skill in the art.

In an embodiment, the oral dosage form may include one or more polycarboxylic acids. Polycarboxylic acids include organic compounds that have two or more carboxyl (—COOH) groups and from 2 to 9 carbon atoms in a chain or ring to which the carboxyl groups are attached. The carboxyl groups are not included when determining the number of carbon atoms in the chain or ring (e.g., 1,2,3 propane tricarboxylic acid would be considered to be a C₃ polycarboxylic acid containing three carboxyl groups and 1,2,3,4 butanetetracarboxylic acid would be considered to be a C₄ polycarboxylic acid containing four carboxyl groups). C₂-C₉ polycarboxylic acids include, but are not limited to aliphatic, aromatic, and alicyclic acids, either saturated or olefinically unsaturated, with at least two carboxyl groups per molecule. In some embodiments, aliphatic polycarboxylic acids may include a hydroxyl group attached to a carbon atom alpha to a carboxyl group (an α-hydroxy polycarboxylic acid). α-hydroxy polycarboxylic acids include citric acid (also known as 2-hydroxy-1,2,3 propane tricarboxylic acid) and tartaric acid.

Examples of specific polycarboxylic acids include, but are not limited to, oxalic acid, malonic acid, succinic acid, glutaric acid, adipic acid, maleic acid, fumaric acid, malic acid, pimelic acid, nonanedioic acid, dodecanedioic acid, octanedioic acid, phthalic acid, isophthalic acid, terephthalic acid, citraconic (methylmaleic acid), citric acid, tartaric acid, itaconic acid (methylenesuccinic acid), 1,2,3 propane tricarboxylic acid, transaconitic acid (trans-1-propene-1,2,3-tricarboxylic acid), 1,2,3,4-butanetetracarboxylic acid, all-cis-1,2,3,4-cyclopentanetetracarboxylic acid, mellitic acid (benzenehexacarboxylic acid), oxydisuccinic acid (2,2′-oxybis(butanedioic acid), α-bromoglutaric acid, 3,3-dimethylpentanedioic acid, and 2,4-dicholoropentanedioic acid.

Polycarboxylic acids may be used as plasticizers. In some embodiments, the combination of a polycarboxylic acid with an enteric polymer may lower the glass transition temperature and/or the viscosity of the resulting mixture. Lowering the glass transition temperature may result in less degration of any thereapuetic agents contained in the mixture of enteric polymer and polycarboxylic acid. Additionally, the addition of a polycarboxylic acid to the enteric polymer may reduce the viscosity of the heated mixture, which leads to more uniform extrusion of the heated mixture.

Bioadhesive polymers include polyethylene oxide, KLUCEL (hydroxypropylcellulose), CARBOPOL, polycarbophil, GANTREZ, Poloxamer, and combinations thereof, and others known to one of ordinary skill in the art.

Retardants are agents that are insoluble or slightly soluble polymers with a Tg above 45° C., or above 50° C. before being plasticized by other agents in the formulation including other polymers and other excipients needed for processing. The excipients include waxes, acrylics, cellulosics, lipids, proteins, glycols, and the like.

Exemplary pore formers include water soluble polymers such as polyethylene glycol, propylene glycol, and povidone; binders such as lactose, calcium sulfate, calcium phosphate and the like; salts such as sodium chloride, magnesium chloride and the like, poloxamers and combinations thereof and other similar or equivalent materials which are widely known in the art.

Poloxamers are triblock copolymers composed of poly(ethylene oxide) (PEO)-poly(propylene oxide) (PPO), poly(ethylene oxide) (PEO) in the configuration:

HO(PEO)ₐ(PPO)_{b}(PEO)ₐH

Poloxamers, also sold under the name Pluronics and Lutrol (BASF Corporation), are crystalline or semi-crystalline materials having molecular weight ranging from about 2,000 to about 20,000 daltons. The molecular weight of pharmaceutical grade poloxamers ranges from about 7,000 to 18,000 daltons.

| Poloxamer | | Substitution | | |
|---|---|---|---|---|
| Type | Physical Form | a | b | Avg. MW |
| 188 | Solid | 79 | 28 | ~7,600-9,000 |
| 237 | Solid | 64 | 37 | ~6,800-9000 |
| 338 | Solid | 141 | 44 | ~12,000-17,000 |
| 407 | Solid | 98 | 57 | ~9,000-14,000 |

Examples of poloxamers include, but are not limited to: Pluronic® F-68 (Poloxamer 188), Pluronic® F87 (Poloxamer 237), Pluronic® F108 (Poloxamer 338), Pluronic® F127 (Poloxamer 407, Lutrol F127) and the like. Pluronic® is a registered tradename for BASF Corporation for block copolymers of ethylene oxide and propylene oxide represented by the chemical structure HO(C₂H₄O)ₐ(C₃H₆O)_{b}(C₂H₄O)ₐH wherein for: (a) Pluronic® F-68, a is 80 and b is 27; (b) Pluronic® F87, a is 64 and b is 37; (c) Pluronic® F108, a is 141 and b is 44; and Pluronic® F127, a is 101 and b is 56. The average molecular weights of these block copolymers are 8,400, 7,700, 14,600 and 12,600 for Pluronic® F-68, Pluronic® F-87, Pluronic® F108 and Pluronic® F127, respectively. Poloxomers may be used as pore formers and as plasticizers. In some embodiments, the combination of a poloxamer with an enteric polymer may lower the glass transition temperature and/or the viscosity of the resulting mixture. Lowering the glass transition temperature may result in less degration of any thereapuetic agents contained in the mixture of enteric polymer and poloxamer. Additionally, the addition of a poloxamer to the enteric polymer may reduce the viscosity of the heated mixture, which leads to more uniform extrusion of the heated mixture.

Exemplary osmagents or osmotic agents include organic and inorganic compounds such as salts, acids, bases, chelating agents, sodium chloride, lithium chloride, magnesium chloride, magnesium sulfate, lithium sulfate, potassium chloride, sodium sulfite, calcium bicarbonate, sodium sulfate, calcium sulfate, calcium lactate, d-mannitol, urea, tartaric acid, raffinose, sucrose, alpha-d-lactose monohydrate, glucose, combinations thereof and other similar or equivalent materials which are widely known in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

It should be understood that compounds used as excipients or that are used to modify the oral dosage form, may serve a variety of functions or purposes. Thus, whether a compound named herein is assigned to one or more classifications or functions, its purpose or function should not be considered as being limited to the named purpose or function.

In one embodiment, an oral dosage form includes a progestogen dispersed in an enteric polymer combined with an estrogen. In an embodiment, the oral dosage form may include particles of a progestogen dispersed in an enteric polymer combined with an estrogen. The enteric polymer dispersed progestogen particles and estrogen may be dispersed in a monolithic, solidified enteric polymer. Alternatively, the enteric polymer dispersed progestogen particles and estrogen may be combined to form a tablet using suitable binders. The oral dosage form may include between about 0.5 mg to about 50 mg of a progestogen and between about 0.01 mg to about 1.0 mg of an estrogen.

The enteric polymer dispersed progestogen particles may be formed by mixing together one or more enteric polymers, the progestogen, and one or more optional plasticizers or excipients. The enteric polymer may represent between about 20% to about 99.9% of the mixture. The resulting mixture is heated. Preferably, the mixture is heated to a temperature above the glass transition point of the enteric polymer, as modified any plasticizers or excipients that, optionally, have been added to the mixture. The resulting heated mixture is allowed to solidify as a solid mass. The solid mass may be used to form the enteric polymer dispersed progestogen particles.

The mixture of the enteric polymer, progestogen, estrogen derivative and any optional plasticizers or excipients can be formed by any suitable means. Well-known mixing means known to those skilled in the art include dry mixing, dry granulation, wet granulation, melt granualation, high shear mixing, low shear mixing, and supercritical fluid processes.

Granulation generally is the process wherein particles of powder are made to adhere to one another to form granules, typically in the size range of 0.2 to 4.0 mm. Granulation methods are generally preferred in pharmaceutical formulations because they produce relatively homogeneous mixing of different sized particles.

A dry granulation process is used to form granules without using a liquid solution. Dry granulation may be conducted on a press using slugging tooling or on a roller compactor commonly referred to as a chilsonator.

Wet granulation involves forming granules using a granulating fluid or wetting agent that is subsequently removed by drying. For progestogens and enteric polymers, a wetting fluid may be an organic solvent having a boiling point of less than about 100° C. Examples of suitable solvents include, but are not limited to, acetone, methanol, ethanol, ethyl ether, ethyl acetate, chlorinated solvents, or mixtures thereof. The enteric polymer and the solubilized or suspended progestogen may be introduced into a granulator. Granulators can be low shear, medium shear, or high shear. Shear is the amount of mechanical force of the granulator. A low-shear granulator uses very little mechanical force to combine powders and binding solution. The fluid-bed granulator, the most commonly used low-shear granulator, uses a high volume of air flow to elevate powders in a chamber while the solution/suspension that contains the active agent(s) is sprayed onto the enteric polymer particles to form a light bond. A fluid-bed granulator does not impart mechanical energy but instead relies on the powder characteristics and the binding solution to form the lightly held powders into granules. After the granulation process is completed, the resulting mixture may be dried to remove at least a portion of the solvent.

In an alternate embodiment, an organic solvent having a boiling point that is greater than 100° C. may be used in a wet granulation process. Examples of such solvents that are suitable for use with progestogens include, but are not limited to: poloxamers, glycerin, triethyl citrate, dibutyl sebacate, triacetin, olive oil, propylene glycol, castor oil, or combinations thereof. The enteric polymer and the solubilized or suspended progestogen may be introduced into a granulator. The resulting mixture may be used directly to form an oral dosage form, without a drying step. The solvent used to dissolve/suspend the mixture may be incorporated into the oral dosage form during subsequent processing.

In an alternate embodiment, an organic solvent having a boiling point that is greater than 100° C. may be used to introduce the progestogen into an extruder. Thus, an enteric polymer may be introduced into an extruder and heated to a temperature at or above the glass transition temperature of the enteric polymer. The solubilized or suspended progestogen may be introduced into the extruder after the enteric polymer is heated. In one embodiment, the solubilized or suspended progestogen may be introduced at a point proximate to the exit of the extruder to minimize the amount of time the active ingredients are exposed to the heated polymer. The solvent used to dissolve/suspend the mixture may be incorporated into the oral dosage form during the extrusion process.

Melt granulation is a process in which powders are transformed into solid aggregates or agglomerates while being heated. It is similar to wet granulation except that a binder acts as a wetting agent only after it has melted. All of these and other methods of mixing pharmaceutical formulations are well-known in the art.

In certain embodiments, a mixture of the enteric polymer, progestogen, estrogen derivative and any optional plasticizers or excipients may be formed by producing a mixture of the selected components in a supercritical fluid and removing the supercritical fluid. In some embodiments the supercritical fluid is carbon dioxide or others known in the art.

In some embodiments, the progestogen may be in a micronized form. Micronized progestogens comprise particles having an average particle size of less than about 50 μm. The micronized progestogen may be combined with an enteric polymer to form a mixture suitable for further processing. In some embodiments, the enteric polymer may also be in a micronized from (i.e., enteric polymer particles have an average particle size of less than about 50 μm)

In some embodiments milling of one or more of the components may be performed to reduce or homogenize the particle size of the components. Techniques that may be used for reducing or homogenize the component particles include, but are not limited to, impact milling, attrition milling, knife milling, and direct-pressure milling.

Impact milling occurs when a hard object that applies a blunt force across a wide area hits a particle to fracture it. This milling action may be produced by a rotating assembly that uses blunt or hammer-type blades. Another type of impact mill is a jet mill. A jet mill uses compressed gas to accelerate the particles, causing them to impact against each other in the process chamber. Impact mills can reduce both fine powders and large chunks of friable material down to average particle sizes of 50 μm with mechanical impact mills, and less than 10 μm with jet mills. Mechanical impact mill types include hammermills, pin mills, cage mills, universal mills, and turbo mills.

In attrition milling, nondegradable grinding media continuously contacts the material, systematically grinding its edges down. This milling action is typically produced by a horizontal rotating vessel filled with grinding media and tends to create free-flowing, spherical particles. Attrition mills can reduce materials down to an average particle size of less than 1 μm. One type of attrition mill is the media mill (also called a ball mill).

In knife milling, a sharp blade applies high, head-on shear force to a large particle, cutting it to a predetermined size to create smaller particles and minimize fines. This milling action is produced by a rotating assembly that uses sharp knives or blades to cut the particles. Knife mills can reduce 2-inch or larger chunks or slabs of material down to 250 to 1,200 μm. Mill types include knife cutters, dicing mills, and guillotine mills.

Direct-pressure milling occurs when a particle is crushed or pinched between two hardened surfaces. Two rotating bars or one rotating bar and a stationary plate generally produce this milling action. Direct-pressure mills typically reduce friable materials down to 800 to 1,000 μm. Types include roll mills, cracking mills, and oscillator mills.

Subsequent or simultaneous with mixing, the mixture of enteric polymer, progestogen, and optional plasticizer and excipients is heated to produce a mass sufficiently fluid to permit shaping of the mixture and/or to produce melding of the components of the mixture. The heated mixture is then permitted to solidify as a substantially solid oral dosage form. The mixture can optionally be shaped or cut into suitable sizes during the heating step or during the solidifying step.

For purposes of the present disclosure a mixture is “heated” by applying thermal or mechanical energy to the mixture. In some embodiments, the mixture may be heated to a temperature such that the mixture is partially or substantially completely molten. For instance, in a mixture that includes an enteric polymer, “melting” the mixture may include substantially melting the enteric polymer without substantially melting one or more other materials present in the mixture (e.g., the therapeutic agent and one or more excipients). Generally, a mixture is sufficiently molten, for example, when it can be extruded as a continuous rod, or when it can be subjected to injection molding.

In preferred embodiments, the mixture becomes a homogeneous mixture either prior to or during the heating step.

Methods of heating the mixture include, but are not limited to, hot-melt extrusion, injection molding and compression molding.

Hot-melt extrusion typically involves the use of an extruder device. Such devices are well-known in the art. Such systems include mechanisms for heating the mixture to an appropriate temperature and forcing the heated feed material under pressure through a die to produce a rod, sheet or other desired shape of constant cross-section. Subsequent to, or simultaneous with, being forced through the die, the extrudate can be cut into smaller sizes appropriate for use as an oral dosage form. Any suitable cutting device known to those skilled in the art can be used, and the mixture can be cut into appropriate sizes either while still at least somewhat soft or after the extrudate has solidified. The extrudate may be cut, ground or otherwise shaped to a shape and size appropriate to the desired oral dosage form prior to solidification, or may be cut, ground or otherwise shaped after solidification. In some embodiments, an oral dosage form may be made as a non-compressed hot-melt extrudate.

Under certain conditions, extrusion of a composition may result in “die-swelling,” a phenomenon in which the extrudate swells diametrically after exiting the die. In certain embodiments, die-swelling can be desirable, producing an extrudate having greater porosity and thus accelerated release characteristics. In other embodiments, it can be desirable to avoid die swelling, thereby producing a more solid composition that has slower therapeutic release and/or is slower to dissolve in a solvent such as aqueous ethanol solutions and/or is harder.

Injection molding typically involves the use of an injection-molding device. Such devices are well-known in the art. Injection molding systems force a melted mixture into a mold of an appropriate size and shape. The mixture solidifies as least partially within the mold and then is released.

Compression molding typically involves the use of an compression-molding device. Such devices are well-known in the art. Compression molding is a method in which the mixture is optionally preheated and then placed into a heated mold cavity. The mold is closed and pressure is applied. Heat and pressure are typically applied until the molding material is cured. The molded oral dosage form is then released from the mold.

An oral dosage form produced by a thermal process may exhibit low moisture content. Reduced moisture content of the oral dosage form may improve the stability of the oral dosage form, thus extending the shelf life of the oral dosage form. In one embodiment, the oral dosage form has a moisture content of less than 5%, less than 4%, less than 3%, less than 2%, or less than 1%.

The final step in the process of making oral dosage forms is permitting the oral dosage form as a substantially solid oral dosage form. The oral dosage form may optionally be shaped either prior to solidification or after solidification of the dosage form. Solidification will generally occur either as a result of cooling of the melted mixture or as a result of curing of the mixture however any suitable method for producing a solid dosage form may be used.

In certain embodiments, prior to administration the substantially solid oral dosage form may be cut, ground or otherwise shaped into its final form, or may be allowed to remain in its final molded configuration. Optionally the substantially solid oral dosage form can further include one or more coatings, including polymeric coatings and the like.

In one embodiment, an oral dosage form may include enteric polymer dispersed progestogen particles and particles of an estrogen derivative compressed into a tablet. The tablet form may include between about 0.5 mg to about 50 mg of enteric polymer dispersed progestogen particles and between about 0.01 mg to about 1.0 mg of an estrogen.

The enteric polymer dispersed progestogen particles may be formed by mixing together one or more enteric polymers and the progestogen as described above. The resulting mixture is heated, preferably to a temperature above the glass transition point of the enteric polymer, as modified any plasticizers or excipients that, optionally, have been added to the mixture and formed into a solid mass. The solid mass may be subjected to a grinding and/or milling process to create enteric polymer dispersed progestogen particles. In an alternate method, the resulting heated mixture may be passed through an extruder under conditions such that an extrudate is produced having a length of no more than 1 mm. In an embodiment, the enteric polymer dispersed progestogen particles, produced by either method, have an average particle size of between about 100 μm and about 700 μm, between about 200 μm and about 600 μm, or between about 300 μm and about 400 μm.

The enteric polymer dispersed progestogen particles may be combined with particles of an estrogen to form a tablet. A mixture of enteric polymer dispersed progestogen particles and an estrogen may be formed by using any type of granulation process, such as the processes described above, or other mixing techniques known in the art. In an embodiment, an inactive ingredient, usually referred to as a binder, is added to the mixture of enteric polymer dispersed progestogen particles and estrogen particles to help hold the tablet together and give it strength. A wide variety of binders may be used and are well known in the art. Binders include, but are not limited to: lactose powder, dibasic calcium phosphate, sucrose, corn (maize) starch, microcrystalline cellulose and modified cellulose (for example hydroxymethyl cellulose). After mixing is complete, the tablet formulation may be placed in a tablet press and compressed to form a tablet.

In some embodiments, an ingredient may be added to the tablet formulation to act as a disintegrant that hydrates readily in water to aid tablet dispersion once swallowed. Some binders, such as starch and cellulose, are also excellent disintegrants. Small amounts of lubricants may be added to the tablet formulation, as well. Examples of lubricants are well known in the art and include, but are not limited to: magnesium stearate, stearic acid (stearin), hydrogenated oil, and sodium stearyl fumarate. Lubricants help the tablets, once pressed, to be more easily ejected from the die.

A coating may be formed on the tablet. Examples of coating include enteric coatings, immediate release coatings, or extended release coatings. In some embodiments, an enteric coating may be formed over the oral dosage form. An enteric coating may be formed from a cellulose based enteric polymer, a vinyl based enteric polymer, or an acrylic acid-methacrylic acid based enteric polymer.

In another embodiment, an oral dosage form may include enteric polymer dispersed progestogen particles compressed into a tablet and an estrogen dispersed in a coating layer formed over the tablet. The oral dosage form may include between about 0.5 mg to about 50 mg of the progestogen and between about 0.01 mg to about 1.0 mg of the estrogen dispersed in the coating layer.

The enteric polymer dispersed progestogen particles may be formed by mixing together one or more enteric polymers and a progestogen as described above. The resulting mixture is heated, preferably to a temperature above the glass transition point of the enteric polymer, as modified any plasticizers or excipients that, optionally, have been added to the mixture. The resulting heated mixture is allowed to solidify as a solid mass and formed into particles as described above. In an embodiment, the enteric polymer dispersed progestogen particles have an average particle size of between about 100 μm and about 700 μm, between about 200 μm and about 600 μm, or between about 300 μm and about 400 μm.

The enteric polymer dispersed progestogen particles may be combined with a binder to form a tablet. Optionally, disintegrants and lubricants may be added to the formulation of drospirenone and binder prior to forming the tablet.

A coating that includes an estrogen may be formed on the tablet. Coatings may be formed from one or more hydrophilic polymers, hydrophobic polymers or enteric polymers. A coating may be formed by a spray coating process, where the polymer and estrogen are dissolved in a solvent and sprayed onto the progestogen containing tablet. The resulting oral dosage form includes enteric polymer dispersed progestogen particles in a tablet form and a coating layer that includes an estrogen dispersed in a polymeric coating material. In an embodiment, the coating may be formed from a mixture of the estrogen in an enteric polymer.

In another embodiment, an oral dosage form includes a progestogen dispersed in an enteric polymer as a monolithic, solidified form and an estrogen formed in a coating layer over the monolithic, solidified form. The oral dosage form may include between about 0.5 mg to about 50 mg of the progestogen and between about 0.01 mg to about 1.0 mg of the estrogen.

A monolithic, solidified enteric polymer that includes a progestogen may be formed by mixing together one or more enteric polymers and a progestogen as described above. The resulting mixture is heated, preferably to a temperature above the glass transition point of the enteric polymer, as modified any plasticizers or excipients that, optionally, have been added to the mixture. The resulting heated mixture is allowed to solidify as a solid mass.

The monolithic, solidified progestogen enteric polymer may be formed in any size suitable for oral administration. In some embodiments, oral dosage forms are roughly cylindrical in shape. In a plane perpendicular to the long axis of the cylinder the roughly cylindrical preferred oral dosage form has a diameter of 5 mm or greater, 6 mm or greater, 7 mm or greater, 8 mm or greater, 9 mm or greater, or 10 mm or greater. Along the long axis of the cylinder the preferred oral dosage form has a length of 1, 2, 3, 4, 5, 6, 7, 8, 9, 10, 11, 12, 13, 14 or 15 mm or greater. Such dosage forms could be formed, for example, by extruding the oral dosage form through a die that is at least 0.5 mm in diameter, 0.6 mm in diameter, 0.7 mm, etc., in diameter and then cutting the extrudate to a length of 1, 2, 3, 4, 5 mm, etc., in length.

A coating that includes an estrogen may be formed on the monolithic, solidified progestogen enteric polymer. Coatings may be formed from one or more hydrophilic polymers, hydrophobic polymers or enteric polymers. A coating may be formed by a spray coating process, where the polymer and estrogen are dissolved in a solvent and sprayed onto the progestogen containing oral dosage form. The resulting oral dosage form includes enteric polymer dispersed progestogen particles in a monolithic, solidified form and a coating layer that includes an estrogen dispersed in a polymeric coating material. In an embodiment, the coating may be formed from a mixture of the estrogen in an enteric polymer.

In another embodiment, an oral dosage form includes a progestogen and an estrogen dispersed in an enteric polymer. In an embodiment, the progestogen and estrogen derivative may be dispersed in a monolithic, solidified enteric polymer. The oral dosage form may include between about 0.5 mg to about 50 mg of a progestogen and between about 0.01 mg to about 1.0 mg of an estrogen dispersed in an enteric polymer.

A monolithic, solidified enteric polymer that includes a progestogen and an estrogen may be formed by mixing together one or more enteric polymers, the progestogen and the estrogen. The mixture may, optionally, include one or more plasticizers or one or more excipients. The enteric polymer may represent between about 20% to about 99.9% of the mixture. The resulting mixture is heated. Preferably, the mixture is heated to a temperature above the glass transition point of the enteric polymer, as modified any plasticizers or excipients that, optionally, have been added to the mixture. The resulting heated mixture is allowed to solidify as a solid mass. The solid mass may be used directly to form the oral dosage form or may, optionally, be shaped into a form for use as an oral dosage form.

The monolithic, solidified progestogen enteric polymer may be formed in any size suitable for oral administration. In some embodiments, oral dosage forms are roughly cylindrical in shape. In a plane perpendicular to the long axis of the cylinder the roughly cylindrical preferred oral dosage form has a diameter of 5 mm or greater, 6 mm or greater, 7 mm or greater, 8 mm or greater, 9 mm or greater, or 10 mm or greater. Along the long axis of the cylinder the preferred oral dosage form has a length of 1, 2, 3, 4, 5, 6, 7, 8, 9, 10, 11, 12, 13, 14 or 15 mm or greater. Such dosage forms could be formed, for example, by extruding the oral dosage form through a die that is at least 0.5 mm in diameter, 0.6 mm in diameter, 0.7 mm, etc., in diameter and then cutting the extrudate to a length of 1, 2, 3, 4, 5 mm, etc., in length.

A coating may be formed on the oral dosage from. Examples of coating include enteric coatings, immediate release coatings, or extended release coatings. In some embodiments, an enteric coating may be formed over the oral dosage form. An enteric coating may be formed from a cellulose based enteric polymer, a vinyl based enteric polymer, or an acrylic acid-methacrylic acid based enteric polymer.

The compositions described herein are suitable for immediate release, controlled release and extended release applications, or combinations thereof, depending on the types of polymers, plasticizers and excipients used and their proportions. Methods for adjusting these characteristics will be apparent to those skilled in the art or can be determined without undue experimentation. For example, immediate release characteristics of the oral dosage forms may be enhanced by the inclusion of hydrophilic polymers, plasticizers and/or excipients to enhance the formation of pores in the oral dosage form, particularly those that begin forming when the oral dosage form is subjected to gastric conditions. Alternatively, immediate release characteristics may be suppressed, for example, by coating the oral dosage form with a suitable enteric coating that does not contain the therapeutic agent. By adjusting variables such as these, a range of release characteristics can be obtained from the oral dosage forms.

In some embodiments, the oral dosage form may be disposed in a capsule. In one embodiment, a monolithic solid oral dosage form may be disposed in a capsule. In other embodiments, enteric polymer dispersed progestogen particles and particles of an estrogen may be disposed in a capsule. Examples of materials that may be used to encapsulate the oral dosage form include, but are not limited to, gelatin capsules, hydroxypropylmethyl cellulose (“HPMC”) capsules, or polysaccharide capsules (e.g., pullulan capsules).

In one embodiment, the oral dosage forms disclosed herein may be used to produce a contraceptive state in a subject. The method of achieving such a state includes administering, to said subject, on each day of at least 21 consecutive days, a daily oral dosage unit, prepared according to any of the embodiments described herein, that includes a combination of a progestogen and an estrogen. The method further includes administering, on each day of 7 or less consecutive days, a daily dosage unit containing no active agent, or alternatively, administering no dosage units for 7 days or less.

In suitable embodiments of this method, the daily dosage units including a combination of a progestogen and an estrogen may be administered for 21, 22, 23 or 24 consecutive days, and the daily dosage units containing no active agent may then be administered for 7, 6, 5 or 4 consecutive days, as appropriate. Furthermore, the daily dosage units including the combination of a progestogen and an estrogen derivative may be administered for 28 consecutive days.

Alternatively, the present method includes administering, on each day of at least 21 consecutive days, a daily dosage unit that includes a combination of a progestogen and an estrogen, followed by administering, on each day of 7 or less consecutive days, a daily dosage unit containing an estrogen alone in an amount of from about 0.01 mg to about 1.0 mg. Oral dosage forms of an estrogen may be formed by hot-melt extrusion, as described above or any of known tableting procedures.

In this alternative method, the daily dosage units that include a combination of a progestogen and an estrogen may suitably be administered for 21, 22, 23 or 24 consecutive days, and the daily dosage units that include an estrogen alone may then be administered for 7, 6, 5 or 4 consecutive days, as appropriate.

Specific oral dosage forms described herein for use as an oral contraceptive include drospirenone and ethinylestradiol in amounts effective to inhibit ovulation in a female subject. An oral dosage form may include, but is not limited to, between about 0.5 mg to about 50 mg, between about 1 mg to 30 mg, or between about 2 mg to 10 mg of drospirenone. An oral dosage form includes, but is not limited to, 1 mg, 1.5 mg, 2 mg, 2.5 mg, 3 mg, 3.5 mg, 4 mg, 4.5 mg, or 5 mg of drospirenone.

In addition to drospirenone, an oral dosage form further includes ethinylestradiol. An oral dosage form may include, but is not limited to, between about 0.01 mg to about 0.05 mg, between about 0.015 mg to 0.04 mg, or between about 0.02 mg to 0.03 mg of ethinylestradiol. An oral dosage form includes, but is not limited to, 0.01 mg, 0.015 mg, 0.02 mg, 0.025 mg, 0.03 mg, 0.035 mg, 0.04 mg, 0.045 mg, or 0.05 mg of ethinylestradiol.

Another specific oral dosage form includes a combination of drospirenone and a nitrated estrogen derivative. The oral dosage form may include, but is not limited to, between about 0.5 mg to about 50 mg, between about 1 mg to 30 mg, or between about 2 mg to 10 mg of drospirenone. An oral dosage form includes, but is not limited to, 1 mg, 1.5 mg, 2 mg, 2.5 mg, 3 mg, 3.5 mg, 4 mg, 4.5 mg, or 5 mg of drospirenone.

In addition to drospirenone, an oral dosage form further includes a nitrated estrogen derivative. The oral dosage form may include, but is not limited to, between about 0.01 mg to about 1.0 mg of a nitrated estrogen derivative. An oral dosage form includes, but is not limited to, 0.01 mg, 0.05 mg, 0.1 mg, 0.2 mg, 0.3 mg, 0.4 mg, 0.5 mg, 0.7 mg, or 1.0 mg of a nitrated estrogen derivative.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Drospirenone was dissolved in acetone and spray/granulated with hydroxypropyl methylcellulose acetate succinate. Citric acid was also added to the mixture as a thermal lubricant. The resulting mixture was dried and added to a hot-melt extruder. The mixture was heated until the hydroxypropyl methylcellulose acetate succinate was sufficiently melted to allow extrusion. The extrudate was cooled, and the solidified mass was pulverized into a powder. The drospirenone—enteric polymer powder was formed into a tablet having a weight of about 80 mg. The tablet was spray coated with a solution that includes an immediate release coating polymer (hydroxypropyl cellulose, hydroxypropyl methylcellulose, or polyvinylacetate) and ethinylestradiol.

### Example 2

Drospirenone and ethinylestradiol were dissolved in acetone and spray/granulated with hydroxypropyl methylcellulose acetate succinate. Citric acid was also added to the mixture as a thermal lubricant. The resulting mixture was dried and added to a hot-melt extruder. The mixture was heated until the hydroxypropyl methylcellulose acetate succinate was sufficiently melted to allow extrusion. The extrudate was cooled, and the solidified mass was pulverized into a powder. The drospirenone/ethinylestradiol enteric polymer powder was formed into a tablet having a weight of about 80 mg.

### Example 3

A formulation that included hydroxypropyl methylcellulose acetate succinate (HPMCAS; Aqoat HPMCAS-LG, Shin-Etsu Chemical Co., Ltd., Japan) (79% w/w), drospirenone (1%, w/w), triethyl citrate (10%, w/w) and citric acid (10%, w/w) was extruded at a final temperature of 140° C. on a twin-screw extruder. A solid molecular dispersion of drospirenone was obtained. Drospirenone crystals could not be detected when the extrudate was observed under 100× magnification. After cooling, the solidified mass was processed in a Fitzmill® (The Fitzpatrick Company, Elmhurst, Ill.) and the formed particulates passed through a 60-mesh sieve to obtain progestogen-enteric polymer particles.

### Example 4

A formulation that included hydroxypropyl methylcellulose acetate succinate (Aqoat HPMCAS-LG) (79% w/w), drospirenone (1%, w/w), triethyl citrate (10%, w/w) and Poloxamer 407 (BASF, Pluronic F127) (10%, w/w) was extruded at a final temperature of 140° C. on a twin-screw extruder. A solid molecular dispersion of drospirenone was obtained. Drospirenone crystals could not be seen when the extrudate was observed under 100× magnification. After cooling, the solidified mass was processed in a Fitzmill® and the formed particulates passed through a 60-mesh sieve to obtain progestogen-enteric polymer particles.

### Example 5

In order to minimize the time in which drospirenone is within the extruder, it may be fed downstream to reduce exposure time. The formulation outlined in Table 1 was prepared using a twin-screw extruder at 120° C. as indicated.

TABLE 1

| Compound | % (w/w) | Material Feeding |
|---|---|---|
| AQOAT ® LF | 55 | Upstream |
| Poloxamer 407 | 10 | |
| AQOAT ® LF | 24.5 | Downstream |
| Drospirenone | 0.5 | |
| Triethyl Citrate | 10 | Injection Port |

A solid particle dispersion of drospirenone was obtained. Drospirenone crystals could not be seen visually, but could be detected when the extrudate was observed under 100× magnification. After cooling, the solidified mass was processed in a Fitzmill® and the formed particulates passed through a 60-mesh sieve to obtain progestogen-enteric polymer particles.

### Example 6

Hydroxypropyl methylcellulose acetate succinate (Aqoat HPMCAS-LG) (79%, w/w) was processed in a Fitzmill® to produce particulate HPMCAS-LG having an average particle size of less than about 600 μm. The milled HPMCAS-LG, drospirenone (1% w/w) and Poloxamer 407 (10%, w/w) were blended in a high shear mixer. The blended mixture was granulated with triethyl citrate (10%, w/w) in a high shear mixer and sieved through a 12 mesh screen. The resulting granulated mixture was heated in a twin-screw extruder to a temperature of about 110° C. and extruded. After cooling, the solidified mass was processed in a Fitzmill® and the formed particulates passed through a 60-mesh sieve to obtain progestogen-enteric polymer particles.

### Example 7

Hydroxypropyl methylcellulose acetate succinate (Aqoat HPMCAS-LG) was milled to an average particle size of less than about 600 μm. The milled HPMCAS-LG (89.6%, w/w), Klucel® Pharm Hydroxypropylcellulose (HPC) grade EXF (D50 typically 60-100 μm, molecular weight of 80 kDa, Aqualon, Hercules Inc, Wilmington, Del., “HPC-EXF”) (5%) and drospirenone (5.4% w/w) were blended in a high shear mixer. The blended mixture was granulated using 99% isopropyl alcohol. The resulting granulated mixture was heated in a twin-screw extruder to a temperature of about 140° C. and extruded. After cooling, the solidified mass was processed in a Fitzmill® to form progestogen-enteric polymer particles.

### Example 8

Hydroxypropyl methylcellulose acetate succinate (Aqoat HPMCAS-LG) was milled to an average particle size of less than about 600 μm. The milled HPMCAS-LG (84.6%, w/w), Klucel® Pharm Hydroxypropylcellulose (HPC) grade EXF (D50 typically 60-100 μm, molecular weight of 80 kDa, Aqualon, Hercules Inc, Wilmington, Del., “HPC-EXF”) (10%) and drospirenone (5.4% w/w) were blended in a high shear mixer. The blended mixture was granulated using 99% isopropyl alcohol. The resulting granulated mixture was heated in a twin-screw extruder to a temperature of about 140° C. and extruded. After cooling, the solidified mass was processed in a Fitzmill® to form progestogen-enteric polymer particles.

### Example 9

Hydroxypropyl methylcellulose acetate succinate (Aqoat HPMCAS-LG) was milled to an average particle size of less than about 600 μm. The milled HPMCAS-LG (87.5%, w/w), Klucel® Pharm Hydroxypropylcellulose (HPC) grade EXF (D50 typically 60-100 μm, molecular weight of 80 kDa, Aqualon, Hercules Inc, Wilmington, Del., “HPC-EXF”) (5%) and drospirenone (7.5% w/w) were blended in a high shear mixer. The blended mixture was granulated using 99% isopropyl alcohol. The resulting granulated mixture was heated in a twin-screw extruder to a temperature of about 140° C. and extruded. After cooling, the solidified mass was processed in a Fitzmill® to form progestogen-enteric polymer particles.

### Example 10

Hydroxypropyl methylcellulose acetate succinate (Aqoat HPMCAS-LG) was milled to an average particle size of less than about 600 μm. The milled HPMCAS-LG (82.5%, w/w), Klucel® Pharm Hydroxypropylcellulose (HPC) grade EXF (D50 typically 60-100 μm, molecular weight of 80 kDa, Aqualon, Hercules Inc, Wilmington, Del., “HPC-EXF”) (10%) and drospirenone (7.5% w/w) were blended in a high shear mixer. The blended mixture was granulated using 99% isopropyl alcohol. The resulting granulated mixture was heated in a twin-screw extruder to a temperature of about 140° C. and extruded. After cooling, the solidified mass was processed in a Fitzmill® to form progestogen-enteric polymer particles.

### Example 11

The holt-melt extruded drospirenone particles obtained in Example 3 were mixed with ethinylestradiol to form a tablet. Hydroxypropyl cellulose and water were mixed using an overhead mixer. The resulting aqueous mixture of hydroxypropyl cellulose was added to a fluid bed granulator along with microcrystalline cellulose and micronized ethinylestradiol. The combined components were granulated and passed through a 50-mesh sieve to produce a granulated mixture having an average particle size of less than about 300 μm. The granulated mixture was combined with the drospirenone particles, crospovidone and magnesium stearate and mixed using a V-Shell blender. The blended mixture of drospirenone particles and ethinylestradiol was introduced into a tablet press and compressed into a tablet. The tablet was coated with Eudragit L30-D55 in a tablet coater.

### Example 12

The holt-melt extruded drospirenone particles obtained in Example 9 were mixed with ethinylestradiol to form a tablet. Hydroxypropyl cellulose, ethinylestradiol, isopropanol and water were mixed using an overhead mixer. The resulting aqueous mixture was added to a fluid bed granulator along with microcrystalline cellulose. The combined components were granulated and passed through a 50-mesh sieve to produce a granulated mixture having an average particle size of less than about 300 μm. The granulated mixture was combined with the drospirenone particles, crospovidone and magnesium stearate and mixed using a V-Shell blender. The blended mixture of drospirenone particles and ethinylestradiol was introduced into a tablet press and compressed into a tablet. The tablet was coated with Eudragit L30-D55 in a tablet coater.

### Example 13

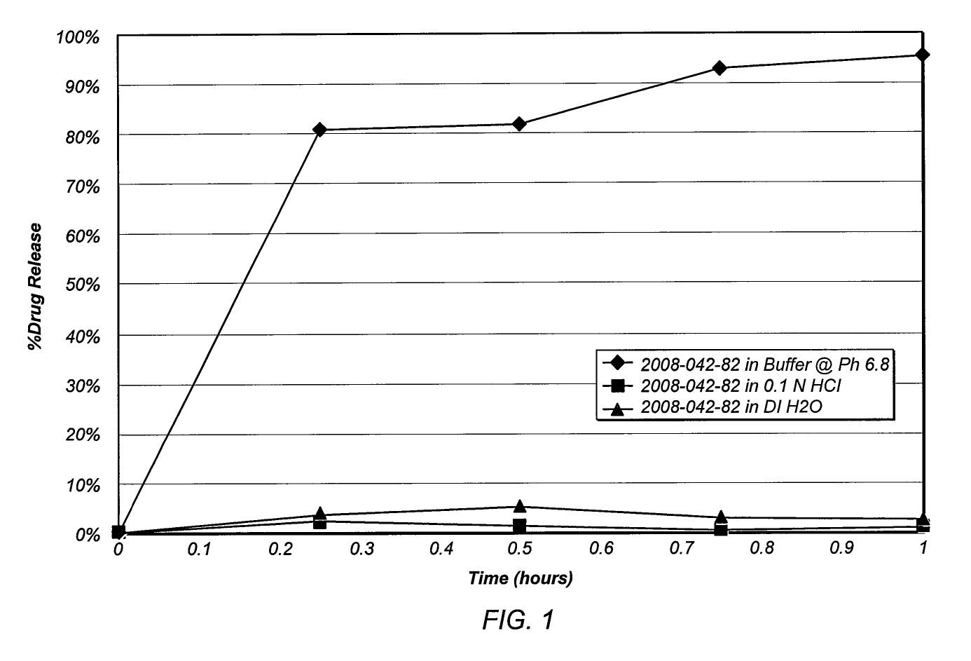

Hydroxypropyl methylcellulose acetate succinate (HPMCAS; Aqoat HPMCAS-LG, Shin-Etsu Chemical Co., Ltd., Japan) was processed in a Fitzmill® (The Fitzpatrick Company, Elmhurst, Ill.) to produce particulate HPMCAS-LG having an average particle size of less than about 600 μm. The milled HPMCAS-LG, drospirenone and HPC-EXP were blended in a high shear mixer. The concentration of each component in the blended mixture was: 89.6% HPMCAS-LG; 5% HPC-EXF; and 5.4% drospirenone. The blended mixture was granulated with isopropyl alcohol in a high shear mixer. The resulting granulated mixture was heated in a twin-screw extruder to a temperature of about 140° C. and extruded. After cooling, the solidified mass was processed in a Fitzmill® and the formed particulates passed through a sieve such that the obtained hot-melt extruded drospirenone particles had an average particle size of less than 50 μm. The dissolution profile of the drospirenone particles in D.I. water, 0.1N HCl, and pH 6.8 buffer is depicted in FIG. 1

### Example 14

Holt-melt extruded drospirenone particles were mixed with ethinylestradiol to form a tablet. The compounds listed in Table 2 were used to form the tablet.

TABLE 2

| | Compound | % (w/w) | mg/tablet |
|---|---|---|---|
| Drospirenone | AQOAT ® LG | 55.78 | 50.20 |
| Extrudate | HPC-EXF | 3.11 | 2.80 |
| | Drospirenone | 3.33 | 3.00 |
| | Total Extrudate | 62.22 | 56.00 |
| Ethinyl Estradiol | Avicel PH-101 | 21.08 | 18.97 |
| Granulation | Ethinyl Estradiol | 0.03 | 0.03 |
| | HPC-EXF | 1.11 | 1.00 |
| | Total Granulation | 22.22 | 20.00 |
| Extragranular | Crospovidone XL | 5.33 | 4.80 |
| Excipients | HPC-EXF | 9.56 | 8.60 |
| | Mg-Stearate | 0.67 | 0.6 |
| | Total Core Tablet | 100.00 | 90.00 |

HPMCAS-LG was processed in a Fitzmill to produce particulate HPMCAS-LG having an average particle size of less than about 600 μm. The milled HPMCAS-LG, drospirenone and HPC-EXF were blended in a high shear mixer. The blended mixture was granulated with isopropyl alcohol in a high shear mixer. The resulting granulated mixture was heated in a twin-screw extruder to a temperature of about 120° C. and extruded. After cooling, the solidified mass was processed in a Fitzmill® and the formed particulates passed through a sieve such that the obtained hot-melt extruded drospirenone particles had an average particle size of less than 50 μm.

HPC-EXF was added to a fluid bed granulator along with Avicel PH-101 (FMC Biopolymers, Philadelphia, Pa.) and ethinylestradiol. The combined components were granulated and passed through a 50-mesh sieve to produce a granulated mixture having an average particle size of less than about 300 μm. The granulated mixture was combined with the drospirenone particles, HPC-EXF, Crospovidone XL (Polyplasdone XL, ISP, Wayne, N.J.) and magnesium stearate and mixed using a V-Shell blender. The blended mixture of drospirenone particles and ethinylestradiol was introduced into a tablet press and compressed into a tablet. The tablet was coated with a hydroxypropyl methylcellulose coating (Opadry II, Colorcon, West Point, Pa.) to a weight gain of about 5%. These tablets were then coated with Eudragit® L30-D55 to approximately 3%, 5%, or 8% weight gain.

### Example 15

Holt-melt extruded drospirenone particles were mixed with ethinylestradiol to form a tablet. The compounds listed in Table 3 were used to form the tablet.

TABLE 3

| | Compound | % (w/w) | mg/tablet |
|---|---|---|---|
| Drospirenone | AQOAT ® LG | 55.78 | 50.20 |
| Extrudate | HPC-EXF | 3.11 | 2.80 |
| | Drospirenone | 3.33 | 3.00 |
| | Total Extrudate | 62.22 | 56.00 |
| Ethinyl Estradiol | Avicel PH-101 | 21.08 | 18.97 |
| Granulation | Ethinyl Estradiol | 0.03 | 0.03 |
| | HPC-EXF | 1.11 | 1.00 |
| | Total Granulation | 22.22 | 20.00 |
| Extragranular | Crospovidone XL | 5.33 | 4.80 |
| Excipients | AQOAT ® LF | 9.56 | 8.60 |
| | Mg-Stearate | 0.67 | 0.6 |
| | Total Core Tablet | 100.00 | 90.00 |

HPMCAS-LG was processed in a Fitzmill to produce particulate HPMCAS-LG having an average particle size of less than about 600 μm. The milled HPMCAS-LG, drospirenone and HPC-EXF were blended in a high shear mixer. The blended mixture was granulated with isopropyl alcohol in a high shear mixer. The resulting granulated mixture was heated in a twin-screw extruder to a temperature of about 120° C. and extruded. After cooling, the solidified mass was processed in a Fitzmill® and the formed particulates passed through a sieve such that the obtained hot-melt extruded drospirenone particles had an average particle size of less than 50 μm.

HPC-EXF was added to a fluid bed granulator along with Avicel PH-101 (FMC Biopolymers, Philadelphia, Pa.) and ethinylestradiol. The combined components were granulated and passed through a 50-mesh sieve to produce a granulated mixture having an average particle size of less than about 300 μm. The granulated mixture was combined with the drospirenone particles, AQOAT® LF, Crospovidone XL (Polyplasdone XL, ISP, Wayne, N.J.) and magnesium stearate and mixed using a V-Shell blender. The blended mixture of drospirenone particles and ethinylestradiol was introduced into a tablet press and compressed into a tablet. The tablet was coated with a hydroxypropyl methylcellulose coating (Opadry II, Colorcon, West Point, Pa.) to a weight gain of about 5%. These tablets were then coated with Eudragit L30-D55 to approximately 3%, 5%, or 8% weight gain.

### Example 16

Holt-melt extruded drospirenone particles were mixed with ethinylestradiol to form a tablet. The compounds listed in Table 4 were used to form the tablet.

TABLE 4

| | Compound | % (w/w) | mg/tablet |
|---|---|---|---|
| Drospirenone | AQOAT ® LG | 55.78 | 50.20 |
| Extrudate | HPC-EXF | 3.11 | 2.80 |
| | Drospirenone | 3.33 | 3.00 |
| | Total Extrudate | 62.22 | 56.00 |
| Ethinyl Estradiol | Avicel PH-101 | 21.08 | 18.97 |
| Granulation | Ethinyl Estradiol | 0.03 | 0.03 |
| | HPC-EXF | 1.11 | 1.00 |
| | Total Granulation | 22.22 | 20.00 |
| Extragranular | Crospovidone XL | 5.33 | 4.80 |
| Excipients | Avicel PH-101 | 9.56 | 8.60 |
| | Mg-Stearate | 0.67 | 0.6 |
| | Total Core Tablet | 100.00 | 90.00 |

HPMCAS-LG was processed in a Fitzmill to produce particulate HPMCAS-LG having an average particle size of less than about 600 μm. The milled HPMCAS-LG, drospirenone and HPC-EXF were blended in a high shear mixer. The blended mixture was granulated with isopropyl alcohol in a high shear mixer. The resulting granulated mixture was heated in a twin-screw extruder to a temperature of about 120° C. and extruded. After cooling, the solidified mass was processed in a Fitzmill® and the formed particulates passed through a sieve such that the obtained hot-melt extruded drospirenone particles had an average particle size of less than 50 μm.

HPC-EXF was added to a fluid bed granulator along with Avicel PH-101 (FMC Biopolymers, Philadelphia, Pa.) and ethinylestradiol. The combined components were granulated and passed through a 50-mesh sieve to produce a granulated mixture having an average particle size of less than about 300 μm. The granulated mixture was combined with the drospirenone particles, Avicel PH-101, Crospovidone XL and magnesium stearate and mixed using a V-Shell blender. The blended mixture of drospirenone particles and ethinylestradiol was introduced into a tablet press and compressed into a tablet. The tablet was coated with a hydroxypropyl methylcellulose coating (Opadry II) to a weight gain of about 5%. These tablets were then coated with Eudragit® L30-D55 to approximately 3%, 5%, or 8% weight gain.

### Example 17

Holt-melt extruded drospirenone particles were mixed with ethinylestradiol to form a tablet. The compounds listed in Table 5 were used to form the tablet.

TABLE 5

| | Compound | % (w/w) | mg/tablet |
|---|---|---|---|
| Drospirenone | AQOAT ® LG | 63.45 | 47.59 |
| Extrudate | HPC-EXF | 3.56 | 2.67 |
| | Drospirenone | 4.19 | 3.14 |
| | Total Extrudate | 71.20 | 53.40 |
| Ethinyl Estradiol | Avicel PH-101 | 11.36 | 8.52 |
| Granulation | Ethinyl Estradiol | 0.04 | 0.03 |
| | HPC-EXF | 0.60 | 0.45 |
| | Total Granulation | 12.00 | 9.00 |
| Extragranular | Crospovidone XL | 5.30 | 3.98 |
| Excipients | HPC-EXF | 11.00 | 8.25 |
| | Mg-Stearate | 0.50 | 0.37 |
| | Total Core Tablet | 100.00 | 75.00 |

HPMCAS-LG was processed in a Fitzmill to produce particulate HPMCAS-LG having an average particle size of less than about 600 μm. The milled HPMCAS-LG, drospirenone and HPC-EXF were blended in a high shear mixer. The blended mixture was granulated with isopropyl alcohol in a high shear mixer. The resulting granulated mixture was heated in a twin-screw extruder to a temperature of about 120° C. and extruded. After cooling, the solidified mass was processed in a Fitzmill® and the formed particulates passed through a sieve such that the obtained hot-melt extruded drospirenone particles had an average particle size of less than 50 μm.

HPC-EXF was added to a fluid bed granulator along with Avicel PH-101 (FMC Biopolymers, Philadelphia, Pa.) and ethinylestradiol. The combined components were granulated and passed through a 50-mesh sieve to produce a granulated mixture having an average particle size of less than about 300 μm. The granulated mixture was combined with the drospirenone particles, HPC-EXF, Crospovidone XL and magnesium stearate and mixed using a V-Shell blender. The blended mixture of drospirenone particles and ethinylestradiol was introduced into a tablet press and compressed into a tablet. The tablet was coated with a hydroxypropyl methylcellulose coating (Opadry II) to a weight gain of about 5%. These tablets were then coated with Eudragit® L30-D55 to approximately 5% weight gain.

### Example 18

Holt-melt extruded drospirenone particles were mixed with ethinylestradiol to form a tablet. The compounds listed in Table 6 were used to form the tablet.

TABLE 6

| | Compound | % (w/w) | mg/tablet |
|---|---|---|---|
| Drospirenone | AQOAT ® LG | 46.67 | 35.00 |
| Extrudate | HPC-EXF | 2.67 | 2.00 |
| | Drospirenone | 4.00 | 3.00 |
| | Total Extrudate | 53.33 | 40.00 |
| Ethinyl Estradiol | Avicel PH-101 | 11.36 | 8.52 |
| Granulation | Ethinyl Estradiol | 0.04 | 0.03 |
| | HPC-EXF | 0.60 | 0.45 |
| | Total Granulation | 12.00 | 9.00 |
| Extragranular | Crospovidone XL | 5.33 | 4.00 |
| Excipients | HPC-EXF | 10.00 | 7.50 |
| | Mg-Stearate | 0.50 | 0.38 |
| | Avicel PH-101 | 18.83 | 14.13 |
| | Total Core Tablet | 100.00 | 75.00 |

HPMCAS-LG was processed in a Fitzmill to produce particulate HPMCAS-LG having an average particle size of less than about 600 μm. The milled HPMCAS-LG, drospirenone and HPC-EXF were blended in a high shear mixer. The blended mixture was granulated with isopropyl alcohol in a high shear mixer. The resulting granulated mixture was heated in a twin-screw extruder to a temperature of about 120° C. and extruded. After cooling, the solidified mass was processed in a Fitzmill® and the formed particulates passed through a sieve such that the obtained hot-melt extruded drospirenone particles had an average particle size of less than 50 μm.

HPC-EXF was added to a fluid bed granulator along with Avicel PH-101 (FMC Biopolymers, Philadelphia, Pa.) and ethinylestradiol. The combined components were granulated and passed through a 50-mesh sieve to produce a granulated mixture having an average particle size of less than about 300 μm. The granulated mixture was combined with the drospirenone particles, HPC-EXF, Crospovidone XL, Avicel PH-101 and magnesium stearate and mixed using a V-Shell blender. The blended mixture of drospirenone particles and ethinylestradiol was introduced into a tablet press and compressed into a tablet. The tablet was coated with a hydroxypropyl methylcellulose coating (Opadry II) to a weight gain of about 5%. These tablets were then coated with Eudragit® L30-D55 to approximately 5% weight gain.

### Example 19

HPMCAS-LG (AQOAT® LG) was milled through a 0.050″ screen using a Fitzpatrick DAS06 Fitzmill. The milled HPMCAS-LG (2,100.0 g) was blended with HPC-EXF (120.0 g) and drospirenone (180.0 g) in a GMX-25 high shear mixer (Vector Corporation, Marion, Iowa) for 3 minutes at 300 rpm followed by granulation with 99% isopropyl alcohol (600.0 g). The wet granulation was performed using the GMX-25 high shear mixer equipped with a peristaltic pump and nozzle to introduce the isopropyl alcohol into the blended mixture. The resulting wet granulation was passed through a Quadro Comil (Quadro Engineering, Ontario, Canada) equipped with a 2A250R03758029 screen (a wet mass screen) and a 2A1601173 impeller in order to eliminate agglomerations. Immediately following milling the granulation was divided into two sub-batches and dried in a Strea-1 fluid bed drying system (GEA Pharma Systems—Niro, Inc., Columbia, Md.) at 60-70° C. to a Loss on Drying (“LOD”) at 105° C. of less than 5%. The sub-batches were recombined by mixing in a Bohle blender (L. B. Bohle, LLC, Warminster, Pa.) equipped with a 20 L bowl for 10 minutes at 25 rpm. The dried granulation was milled and mixed using a Comil equipped with a 2A075R03751 screen (0.075 inch screen) and a 2A1612198 impeller at the lowest motor speed.

The dried granulation was extruded using a Leistritz ZSE 18-mm twin screw extruder system (American LEISTRITZ Extruder Corp, Somerville, N.J.) set-up with a 25:1 length/diameter barrel configuration as follows: Open Barrel (feed); Closed Barrel; Closed barrel; Open Barrel (vent); Closed Barrel; Final Melt Plate. A 4.0 mm round, single-bore die and a spacer were attached to the end of the die plate. The extruder was heated to 140° C. and allowed to equilibrate for 30 minutes. The screw design chosen for this process is detailed in Table 7.

TABLE 7

| | Element Function | | | | |
|---|---|---|---|---|---|
| | Feeding | Conveying | Conveying | Conveying | Kneading |
| Length (mm) | 90 | 60 | 60 | 30 | 15 |
| Pitch (mm) | 30 | 20 | 20 | 20 | N/A |
| Degrees (rotation) | N/A | N/A | N/A | N/A | 60 |
| Element Description | GFF-2-30-90 | GFA-2-20-60 | GFA-2-20-60 | GFA-2-20-30 | KB4-2-15/60 RE |
| | Element Function | | | | |
| | Kneading | Conveying | Conveying | Conveying | Conveying |
| Length (mm) | 15 | 60 | 30 | 60 | 30 |
| Pitch (mm) | N/A | 30 | 30 | 20 | 15 |
| Degrees (rotation) | 60 | N/A | N/A | N/A | N/A |
| Element Description | KB4-2-15/60 RE | GFA-2-30-60 | GFA-2-30-30 | GFA-2-20-60 | GFA-2-15-30 |

The dried granulation blend was transferred to a K-Tron twin screw feeder (K-Tron America, Pitman, N.J.) equipped with 12 mm diameter, 20 mm pitch screws and the feed rate was set 1.0 kg/hr. The extrusion process was initiated with an extruder screw speed of 140 RPM.

The single strand of hot-melt extrudate was draw out of the extruder by a conveyor and pelletizer. The conveyor belt speed and the pelletizer feedroll speed were adjusted accordingly in order to draw out extrudate with a diameter suitable for chopping in the pelletizer. All four cooling fans on the conveyor were turned off.

Once pelletized, the extrudate was milled once through a Fitzpatrick DAS06 Fitzmill. Milling included two steps: course milling through a 0.050-inch screen followed by fine milling through a 0.020-inch screen. Milled extrudate with a particle size of less than 300 microns was collected with the use of a Kason vibratory screener (Kason Corporation, Milburn, N.J.). Particles which were greater than 300 microns were again milled through a 0.020″ screen. This was repeated until an adequate amount of extrudate with a particle size of less than 300 microns was collected, the resulting product is herein referred to as the “Drospirenone Process Intermediate.” The yield from each milling pass through the 0.020″ screen as well as the cumulative yield of the Drospirenone Process Intermediate is shown in Table 8.

TABLE 8

| | <300 Microns (g) | >300 Microns (g) |
|---|---|---|
| 1st Pass | 1003.2 | 701.1 |
| 2nd Pass | 310.1 | 365.7 |
| 3rd Pass | 136.6 | 211.9 |
| 4th Pass | 79.2 | 129.9 |
| 5th Pass | 44.8 | 77.3 |
| Total | 1573.9 | |

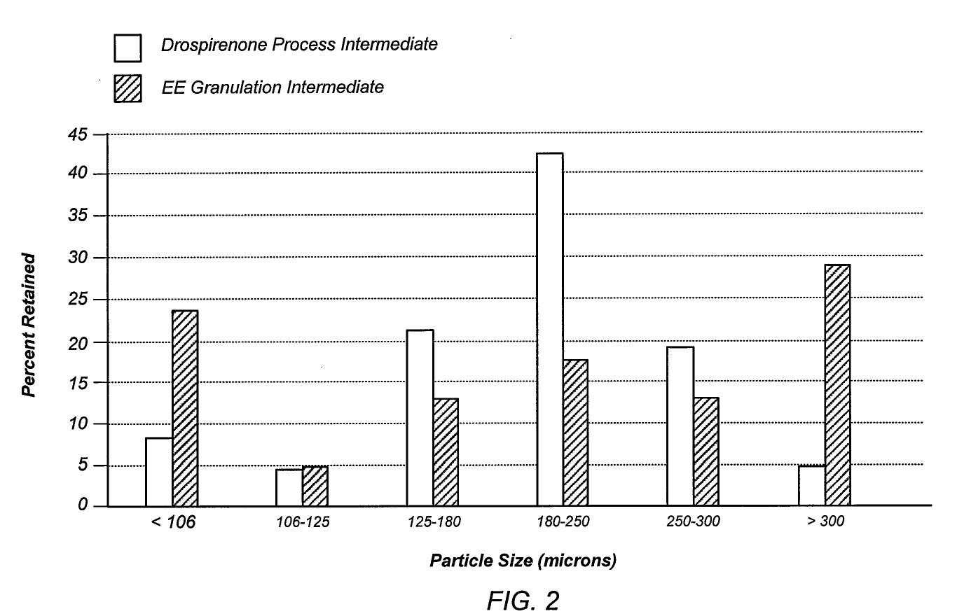

The bulk density of the Drospirenone Process Intermediate was 0.58 and the tapped density of the Drospirenone Process Intermediate was 0.73. The particle size distribution of the Drospirenone Process Intermediate is depicted in FIG. 2. In-process potency and blend uniformity of the Drospirenone Process Intermediate was performed and found to be 97.36% with an RSD of 0.68%.

An ethinylestradiol granulation intermediate (the “EE Granulation Intermediate”) was formed by mixing Avicel PH-101 (2556.0 g) and HPC-EXF (135.0 g) in a GMX-25 high shear mixer for 3 minutes at 330 rpm. Ethinyl Estradiol (9.0 grams) was dissolved into a solution containing sterile water and 99.0% isopropyl alcohol (400.0 grams and 800.0 grams, respectively). This solution was used to granulate the aforementioned blend. Once the reservoir containing the granulation medium was emptied, it was rinsed with additional isopropanol (400.0 grams) which was also used to granulate the blend.

The wet granulation was passed through a Comil equipped with a 2A250R03758029 screen and a 2A1601173 impeller in order to eliminate agglomerations. Immediately after milling, the granulation was dried in a Strea-1 fluid bed drying system at 60-70° C. to a Loss on Drying (LOD) of less than 2.0% at 105° C. In an alternative process, the wet mass is subdivided for drying and the sub-batches are recombined by mixing in a Bohle blender. The dried granulation was then milled and mixed utilizing a Comil equipped with a 2A018R01530 screen and a 2A1612198 impeller at the lowest motor speed in order to more closely match the particle size of the Drospirenone Process Intermediate.

The bulk density of the EE Granulation Intermediate was 0.42 and the tapped density of the EE Granulation Intermediate was 0.56. The particle size distribution of the EE Granulation Intermediate is depicted in FIG. 2. In process potency and blend uniformity (BU) of the EE Granulation Intermediate was performed and found to be 109.27% with a RSD of 0.73%.

Prior to dispensing materials for the final blend, the amount of Drospirenone Process Intermediate and EE Granulation Intermediate were adjusted accordingly for the in-process potency results (97.36% and 109.27%, respectively). The amount of Avicel PH-101 was also adjusted since it has a dual purpose; it is used for a binder/filler as well as a buffer material which is adjusted to correct for in-process potency testing of both active compounds. The Drospirenone Process Intermediate and EE Granulation Intermediate was charged into a Bohle LM 40 Bin Blender, equipped with a 10 L bowl. Avicel PH-101, HPC-EXF, and Crospovidone XL were subsequently charged through a 20 mesh sieve screen into the blender. The blend was mixed at 25 RPM for 10 minutes. A final mixing of the blend was performed using a Comil equipped with a 2A075R03-751 mesh and a 2A1612198 impeller at the lowest motor speed. The blend was charged back into the Bohle blender and magnesium stearate was subsequently charged through a 20 mesh sieve screen into the blender. The blend was mixed at 25 RPM for 8 minutes and transferred to a secondary container. The bulk and tapped densities of the final blend were determined to be 0.45 g/mL and 0.63 g/mL, respectively.

A Stokes B2 tablet press with a 16-station turret was set up utilizing fourteen sets of 0.2362-inch (6.00 mm) circular, standard cup, concave tablet tooling with dust cups. The final blend was transferred into the hopper of the tablet press. The turret speed was set at 25 rpm and the target tablet weight and hardness were 75.0 mg and 5.0 kp, respectively. Initially, and after every 15 minutes of compression, a sample of ten (10) tablet cores were taken to determine in-process parameters. The average weight and hardness of these samples at each time point is shown in Table 9 Compression continued until the hopper was emptied.

TABLE 9

| | Average Weight | Average Hardness |
|---|---|---|
| Time | (mg) | (kp) |
| (minutes) | (n = 10) | (n = 10) |
| 2 | 73.3 | 3.9 |
| 15 | 76.0 | 4.8 |
| 30 | 75.1 | 5.1 |
| 45 | 75.7 | 5.4 |
| 60 | 75.3 | 5.5 |
| 75 | 74.1 | 5.4 |
| 84 | 73.0 | 5.3 |

Samples were taken from the beginning (time=2 minutes) and end (time=84 minutes) of the tableting run and submitted for content uniformity analysis. Results of the initial content uniformity analysis can be seen in Table 10.

TABLE 10

| | Drospirenone | | Ethinyl Estradiol | |
|---|---|---|---|---|
| | Beginning | | Beginning | End |
| Sample | (t = 2 min) | End (t = 84 min) | (t = 2 min) | (t = 84 min) |
| 1 | 88.9% | 91.8% | 93.08% | 86.66% |
| 2 | 91.3% | 92.2% | 92.55% | 91.40% |
| 3 | 91.9% | 87.8% | 83.63% | 88.13% |
| 4 | 91.0% | 137.0% | 85.32% | 134.28% |
| 5 | 90.0% | 89.7% | 91.09% | 85.17% |
| 6 | 90.8% | 89.9% | 87.65% | 85.22% |
| 7 | 91.4% | 90.2% | 87.34% | 91.38% |
| 8 | 90.3% | 91.1% | 84.85% | 95.24% |
| 9 | 90.4% | 94.8% | 96.13% | 80.59% |
| 10 | 92.0% | 90.5% | 87.03% | 87.22% |
| Average | 90.8% | 90.9% | 88.9% | 87.9% |
| RSD | 1.01% | 2.13% | 4.63% | 4.90% |

Tablet cores were then coated with a base coating to no less than 3.0% weight gain using Opadry II (HPMC based) with a Vector LCDS-3 coating system (Vector Corporation, Marion, Iowa). A functional enteric coating containing Eudragit L30 D-55 was applied to no less than 5.0% polymer weight gain using the Vector LCDS-3 coating system. The theoretical formulation for the enteric-coated tablets is listed in Table 11.

TABLE 11

| | Compound | % (w/w) | mg/tablet |
|---|---|---|---|
| Drospirenone | AQOAT ® LG | 46.67 | 35.00 |
| Process Intermediate | HPC-EXF | 2.67 | 2.00 |
| | Drospirenone | 4.00 | 3.00 |
| | Total Extrudate | 53.33 | 40.00 |
| EE Granulation | Avicel PH-101 | 11.36 | 8.52 |
| Intermediate | Ethinyl Estradiol | 0.04 | 0.03 |
| | HPC-EXF | 0.60 | 0.45 |
| | Total Granulation | 12.00 | 9.00 |
| Extragranular | Crospovidone XL | 5.33 | 4.00 |
| Excipients | HPC-EXF | 10.00 | 7.50 |
| | Mg-Stearate | 0.50 | 0.38 |
| | Avicel PH-101 | 18.83 | 14.13 |
| | Total Core Tablet | 100.00 | 75.00 |
| Base Coating | Opadry II | 3.00 | 2.25 |
| Enteric Coating | Eudragit L30 D-55 | 5.00 | 3.75 |
| | Talc | 2.50 | 1.88 |
| | Triethyl Citrate | 0.50 | 0.37 |
| | Total Tablet Weight | 111.00 | 83.25 |

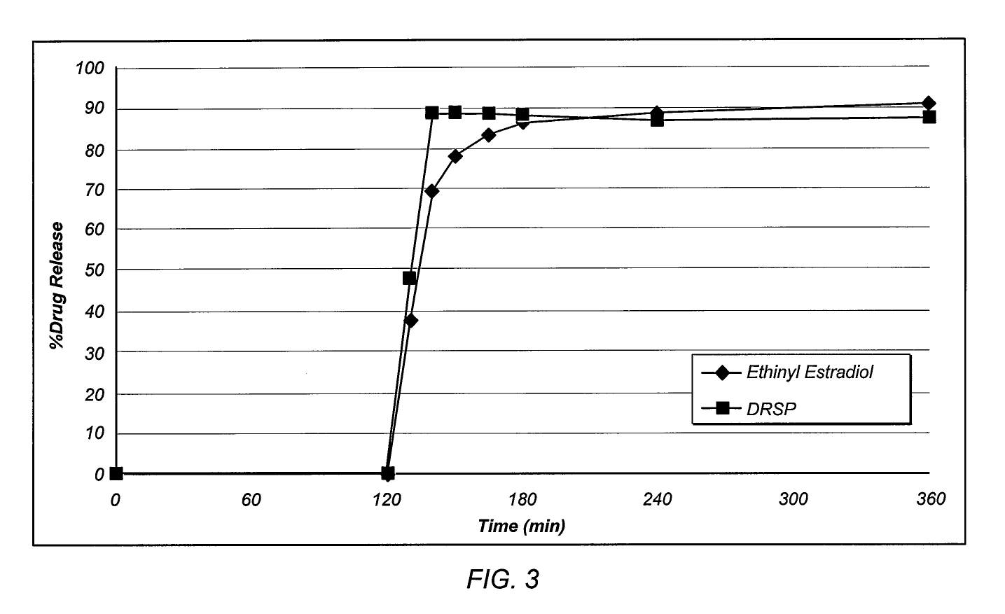

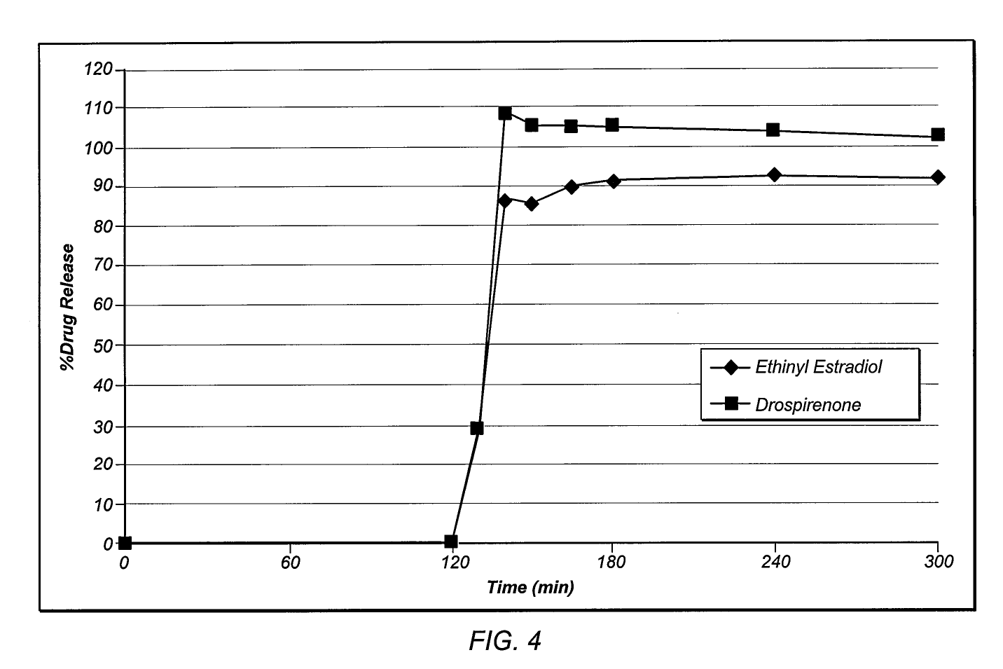

The rate at which the tablets release the drospirenone and ethinylestradiol was determined. Each tablet was placed in 0.1 N HCl and stirred for 2 hours. After this time, the pH of each of the mixtures containing the tablets was adjusted to pH 6.8 with phosphate buffer and stirred for 4 hours using a USP Type II paddle apparatus at 75 rpm and 37° C. The drug release profiles for a first batch of the tablets are shown in FIG. 3. A second test run gave the results listed below in Table 12. A graph of the drug release profiles for the second test run is depicted in FIG. 4.

TABLE 12

| | Average % Drug Release (n = 6) | | | | | | | | |
|---|---|---|---|---|---|---|---|---|---|
| Drug Substance | 5 min | 120 min | 130 min | 140 min | 150 min | 165 min | 180 min | 240 min | 300 min |
| Drospirenone | 0.0 | 0.0 | 29.1 | 108.4 | 105.4 | 105.2 | 105.0 | 103.9 | 102.5 |
| Ethinyl Estradiol | 0.0 | 0.0 | 29.6 | 86.4 | 85.6 | 89.8 | 91.4 | 92.8 | 92.2 |

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. An oral dosage form comprising a progestogen dispersed in an enteric polymer and an estrogen.

2-3. (canceled)

4. The oral dosage form of claim 1, wherein the progestogen is drospirenone.

5. The oral dosage form of claim 1, wherein the estrogen is ethinylestradiol.

6. The oral dosage form of claim 1, wherein the estrogen is a nitrated estrogen derivative.

7. The oral dosage form of claim 6, wherein the nitrated estrogen derivative has the structure: 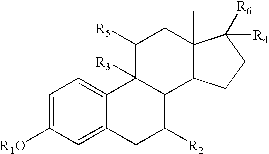
where R₁ is hydrogen, C₁-C₈ alkyl, cycloalkyl, or C₁-C₈ acyl;
R₂ is hydrogen or C₁-C₈ alkyl;
R₃ is hydrogen, hydroxy or C₁-C₈ alkyl;
R₄ is hydrogen or C₁-C₈ alkyl;
where each R₅ and R₆ is, independently, hydrogen or nitrate; and wherein at least one of R₅ and R₆ is a nitrate group.

8-9. (canceled)

10. The oral dosage form of claim 1, wherein the enteric polymer comprises a hydroxypropyl methylcellulose based polymer.

11. The oral dosage form of claim 10, wherein the hydroxypropyl methylcellulose based polymer comprises hydroxypropyl methylcellulose acetate succinate.

12. (canceled)

13. The oral dosage form of claim 1, further comprising one or more hydroxyalkyl celluloses.

14-17. (canceled)

18. An oral dosage form comprising a tablet, wherein the tablet comprises:
progestogen particles, wherein the progestogen particles comprise a progestogen dispersed in an enteric polymer; and
an estrogen.

19-20. (canceled)

21. The oral dosage form of claim 18, wherein the progestogen is drospirenone.

22. The oral dosage form of claim 18, wherein the estrogen is ethinylestradiol.

23. The oral dosage form of claim 18, wherein the estrogen is a nitrated estrogen derivative.

24. The oral dosage form of claim 23, wherein the nitrated estrogen derivative has the structure: 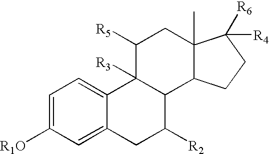
where R₁ is hydrogen, C₁-C₈ alkyl, cycloalkyl, or C₁-C₈ acyl;
R₂ is hydrogen or C₁-C₈ alkyl;
R₃ is hydrogen, hydroxy or C₁-C₈ alkyl;
R₄ is hydrogen or C₁-C₈ alkyl;
where each R₅ and R₆ is, independently, hydrogen or nitrate; and wherein at least one of R₅ and R₆ is a nitrate group.

25-28. (canceled)

29. The oral dosage form of claim 18, wherein the enteric polymer comprises a hydroxypropyl methylcellulose based polymer.

30. The oral dosage form of claim 29, wherein the hydroxypropyl methylcellulose based polymer comprises hydroxypropyl methylcellulose acetate succinate.

31. (canceled)

32. The oral dosage form of claim 18, wherein the progestogen particles further comprise one or more hydroxyalkyl celluloses.

33-34. (canceled)

35. The oral dosage form of claim 18, wherein the tablet further comprises microcrystalline cellulose.

36-38. (canceled)

39. The oral dosage form of claim 18, wherein the oral dosage form further comprises a coating covering at least a portion of the tablet, wherein the coating comprises an enteric polymer.

40-77. (canceled)

78. An oral dosage form comprising a tablet, wherein the tablet comprises:
drospirenone particles, wherein the drospirenone particles comprise drospirenone dispersed in an enteric polymer, wherein the enteric polymer comprises hydroxypropyl methyl cellulose acetate succinate; and
ethinylestradiol;
wherein the tablet is at least partially coated with an enteric polymer.

79. A method of producing a contraceptive state in a subject comprising administering to a subject an oral dosage form comprising an effective amount of a progestogen dispersed in an enteric polymer and an effective amount of an estrogen.

80-172. (canceled)

173. A method of formulating an oral dosage form, comprising:
forming a mixture of an enteric polymer and a progestogen;
heating at least a portion of the enteric polymer to a temperature at or above the glass transition temperature of the enteric polymer to form a heated mixture of enteric polymer and the progestogen;
permitting the heated mixture to solidify as a solid mass comprising the progestogen dispersed in the enteric polymer; and
combining the progestogen dispersed in an enteric polymer with an estrogen to form the oral dosage form.

174-277. (canceled)
